(19) United States
(12) Patent Application Publication
(10) Pub. No.: US 2026/0232428 A1
(43) Pub. Date: Aug. 13, 2026

(54) IMPLANTABLE PROSTHESES FOR TISSUE REGENERATION AND MARKING SURGICAL SITES

(71) Applicant: Davol Inc., Warwick, RI (US)

(72) Inventors: Alice M. Connelli, Warwick, RI (US); Brooke S. Powell, Laurel, MD (US); Bailey M. Florek, Providence, RI (US); Enya Forgaci, Boca Raton, FL (US)

(73) Assignee: Davol Inc., Warwick, RI (US)

(21) Appl. No.: 19/154,491

(22) PCT Filed: Dec. 29, 2023
(86) PCT No.: PCT/US2023/086391

§ 371 Date: Aug. 7, 2025

Related U.S. Application Data
(60) Provisional application No. 63/444,308, filed on Feb. 9, 2023.
(60) Provisional application No. 63/444,315, filed on Feb. 9, 2023.

(30) Foreign Application Priority Data
Jun. 30, 2023 (WO) PCT/US2023/026738

(51) Int. Cl. A61F 2/12; A61F 2/00

(52) U.S. Cl. CPC A61F 2/12; A61F 2/0063; A61F 2002/0068; A61F 2230/0067; A61F 2230/0076; A61F 2250/0098

(57) ABSTRACT

Implantable prostheses and related methods are generally described. In some embodiments, the prosthesis may be a generally ellipsoidal (e.g., spherical) shape, formed of one or more subunits. The subunits may be conical or frustoconical, formed through manipulation of a two-dimensional substrate material. The sidewalls of the subunits may be fixed to one another to enhance the stiffness of the prosthesis. In some embodiments, markers may be used to help distinguish the prosthesis in the implant site through medical imaging modalities (e.g., x-ray imaging). In this way, the implant site, which may also be the site of a recent biopsy and/or a diseased tissue resection, may readily be identified. The prosthesis may be formed of a porous material to induce tissue infiltration, which may facilitate natural tissue regeneration for more natural look and feel of the tissue. Tissue infiltration may result in the prosthesis having an enhanced resistance to migration following implantation.

### FIELD

Disclosed embodiments are related to tissue engineering devices and related methods, and more specifically, to implantable prostheses for soft tissue regeneration and biopsy and lumpectomy site identification.

### BACKGROUND

Soft tissue excision or resection has become an integral and important part of cancer treatment. Tissue can be removed as a biopsy sample to perform diagnostic tests or examinations to determine cytology, histology, presence or absence of chemical substances that act as indicators for disease states, or the presence of bacteria or other microbes. If the biopsy sample indicates malignant (e.g., diseased or cancerous) cells, a surgeon may choose to remove a greater body of tissue to limit the risk of spread and growth of the cells and optimize surgical outcomes.

The removal of a portion of diseased or cancerous cells in breast tissue may be referred to as a lumpectomy, a partial mastectomy, or a mastectomy, which more commonly refers to the removal of the entire breast tissue. Tissue resection or excision can result in an undesirable palpable and/or visible change in the tissue. Thus, patients may seek reconstructive options to fill the void left behind by the procedure, such as injections of fat, autologous tissue, or natural material (e.g., collagen). Alternatively, synthetic materials such as silicone can be employed.

### SUMMARY

In some embodiments, an implantable prosthesis includes a plurality of substantially conical mesh bodies, wherein each of the plurality of substantially conical mesh bodies is connected to at least one other of the plurality of substantially conical mesh bodies, and wherein the substantially conical bodies are arranged to form an ellipsoid.

In some embodiments, a method of forming an implantable prosthesis includes forming a plurality of substantially conical mesh bodies, and connecting each of the conical mesh bodies to at least one other of the other substantially conical mesh bodies to form an ellipsoid.

In other embodiments, an implantable prosthesis includes a plurality of substantially conical bodies, wherein each of the plurality of substantially conical bodies is connected to at least one other of the plurality of substantially conical bodies, and wherein the implantable prosthesis is substantially mechanically isotropic.

In other embodiments, an implantable prosthesis includes a plurality of substantially conical bodies, each conical body including a sidewall defining a cone shape, wherein the sidewall of each substantially conical body is connected to the sidewall of an of at least one other adjacent substantially conical body.

In other embodiments, a method of forming an implantable prosthesis includes forming a plurality of substantially conical bodies, each conical body including a sidewall defining a cone shape, and connecting the sidewall of each substantially conical body to the sidewall of an of at least one other adjacent substantially conical body.

In other embodiments, an implantable prosthesis includes a plurality of mesh bodies, wherein each mesh body is connected to another mesh body, wherein at least some of the mesh bodies include a first mesh portion connected to a second mesh portion, wherein the first portion is arranged inside a volume defined by the second mesh portion.

In other embodiments, a method of forming an implantable prosthesis includes forming a plurality of mesh bodies, arranging a first mesh portion of at least some of the mesh bodies inside a volume defined by a second mesh portion, connecting the first mesh portion to the second mesh portion, and connecting each of the mesh bodies to another mesh body.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1B depict an implantable prosthesis according to some embodiments;

FIG. 2A depicts a top view of the conical subunit of an implantable prosthesis, according to some embodiments;

FIG. 2B depicts an isometric view of the conical subunit of FIG. 2A, according to some embodiments;

FIG. 3 depicts a conical subunit of an implantable prosthesis according to some embodiments;

FIGS. 4A-4D depict conical subunits of implantable prostheses according to other embodiments;

FIGS. 5A-5B depict various views of an implantable prosthesis according to some embodiments;

FIG. 6A depicts an implantable prosthesis according to other embodiments;

FIG. 6B depicts the implantable prosthesis of FIG. 6A along line 6B-6B;

FIGS. 7A-7C depict three implantable prostheses according to other embodiments still;

FIGS. 8A-8B depict various views of an ellipsoidal implantable prosthesis according to some embodiments;

FIGS. 9A-9F depict conical subunits of the implantable prosthesis of FIGS. 8A-8B, according to some embodiments;

FIGS. 10A-10E depict an assembly process for an implantable prosthesis according to some embodiments;

FIGS. 11A-11B depict a compressive testing system for implantable prostheses, according to some embodiments;

FIG. 12 depicts a partial assembly process for an implantable prosthesis according to some embodiments;

FIGS. 13A-13D depict an assembly process for an implantable prosthesis according to some embodiments;

FIG. 14 depicts an implantable prosthesis according to other embodiments;

FIGS. 15A-15D depict an assembly process for an implantable prosthesis according to other embodiments still;

FIGS. 16A-16B depict an implantable prosthesis according to other embodiments still;

FIGS. 17A-17B depict an implantable prosthesis according to other embodiments still;

FIGS. 18A-18B depicts an implantable prosthesis according to other embodiments still;

FIG. 19 depicts an implantable prosthesis according to other embodiments still;

FIGS. 20A-20D depict an assembly process for an implantable prosthesis according to other embodiments still;

FIG. 21 shows exemplary local tissue response data from experimental implantation of an implantable prosthesis according to some embodiments; and

FIG. 22 shows exemplary cellular response data from experimental implantation of an implantable prosthesis according to some embodiments.

### DETAILED DESCRIPTION

The removal of natural tissue at a tissue removal site, which can occur during therapeutic treatment, can result in external dimpling or disfigurement, which may affect both the look and palpability of the natural tissue. Conventional tissue reconstruction using autologous fat or soft natural material fillers can yield undesirable results due to the lack of mechanical stiffness of the injectable materials, which are unable to support the tissue of the implant site. Furthermore, such materials may slow down tissue ingrowth within the tissue removal site, prolonging the healing and reconstruction process. Alternative options such as silicone may be sufficiently stiff enough to support the surrounding tissue, but may substantially limit the potential for tissue ingrowth indefinitely. Furthermore, fluid or material and synthetic filler materials can be incompatible with cancer treatments such as radiation therapy. Such treatments may result in material leeching. Thus, the Inventors have recognized a need for a soft tissue prosthesis which may simultaneously exhibit mechanical properties to support the anatomy of the implant site, while also enabling rapid tissue ingrowth.

In addition, in cancer treatment, radiation therapy is often performed following the removal of a tumor to destroy remaining cancer cells, and lower the risk of cancer recurrence. However, the Inventors have recognized that delineating the tissue margins of a tumor cavity postoperatively for radiation therapy can be difficult. Traditionally, clinicians have relied upon the surgical scar site or the presence of seroma to identify the site for radiation therapy and the radiation target volume. However, these identification methods are not the most accurate, and cannot only reduce the effectiveness of radiation therapy but can also increase the chances that healthy tissue surrounding the cavity will be damaged. Correctly locating the margins of a tumor resection cavity can also be extremely difficult because the cavity may have an irregular shape, and in some tissues the shape can change over time. For example, the tumor cavity may grow or shrink during respiration, or may even change in size and shape as a result of continued radiation therapy treatments. Markers are also used in instances where the biopsy results are normal (e.g., benign) to provide information about biopsy history in follow-up tests (e.g., mammograms).

In some cases, clinicians often use marker devices to better define the location of the cavity, and provide a clearer target for external radiation beam treatment. A marker device is a marker or set of markers placed in an imaging field as a point of reference, which is conventionally formed of surgical alloys such as titanium alloys, including shape memory alloys. The markers are typically small metal objects, which are distinct from surrounding tissue through a variety of imaging modalities (e.g., x-ray), but may suffer from a tendency to migrate following implantation, resulting in an accurate reading of the biopsy or lumpectomy site. Thus, the Inventors have also recognized a need for a biopsy or lumpectomy site marker to guide radiologic targeting in therapeutic and imaging applications.

In view of the above, the Inventors have recognized the benefits of an implantable prosthesis for soft tissue reconstruction in applications such as surgically excised or resected tissue (e.g., in a lumpectomy procedure) and/or for natural soft tissue volume loss. The prosthesis may have mechanical and geometric properties akin to natural tissue to mimic the natural feel of tissue. The prosthesis may further serve as a scaffold for tissue infiltration, to allow natural (or otherwise) tissue to grow within the prosthesis in order to retain mechanical properties akin to natural tissue without being significantly palpable externally. Tissue in-growth into the void space of the resection or excision cavity may also have an added benefit of an improved cosmetic outcome, as well as a resistance to migration. The prosthesis may further serve as an indicator of a biopsy and/or tissue resection (e.g., lumpectomy) site. The prosthesis may be visible using one or more medical imaging systems, to enable external detection of the site for therapeutic and imaging applications. The prosthesis may have the benefit of a reduced clinical target volume in radiotherapy and improve cosmetic outcomes following lumpectomy. However, instances in which different benefits are offered by the systems and methods disclosed herein are also possible.

In some embodiments, an implantable prosthesis may be a three-dimensional implant formed of an assembly of subunits. Each subunit may be formed of a two-dimensional substrate, which may be shaped from the two-dimensional configuration into a three-dimensional configuration. In some embodiments, the two-dimensional substrate may be a generally c-shaped substrate with a cutout, as will be described in greater detail below. The two-dimensional c-shaped substrate may be arranged into a three-dimensional shape by fixing the two ends of the c-shaped substrate together. In this way, a cone (or frustoconical or conical frustum) shape having sidewalls may be formed. It should be appreciated that the two-dimensional substrate may have any shape to facilitate its transformation into a three-dimensional subunit body. In some embodiments, the two ends of the c-shaped substrate may be fixed together using permanent means (e.g., welding), whereas in other embodiments, the two ends may be fixed together using temporary means (e.g., a fastener such as a staple). The sidewalls of the three-dimensional subunit may then be fixed to sidewalls of one or more other subunits to form the three-dimensional implantable prosthesis. For example, twelve conical frustum subunits may be arranged fixed to one another to form a generally ellipsoidal shape.

The Inventors have recognized the benefits associated with implantable prostheses which balance mechanical properties to support surrounding anatomy with high rates of tissue infiltration. A highly stiff and dense implantable prosthesis would support the surrounding tissue without providing natural palpability or facilitating tissue ingrowth. On the other hand, the lack of prosthesis may induce natural tissue ingrowth, but may exhibit a dip or disfigurement at the tissue removal site. Thus, the implantable prostheses of the present disclosure may exhibit both mechanical isotropy and large void spaces to enable tissue ingrowth. In this way, the prostheses may provide sufficient and isotropic mechanical support to the implant site while still enabling rapid tissue ingrowth.

The construction of the implantable prostheses using subunits may enable greater tissue infiltration through the prosthesis when compared to a non-porous or solid prosthesis. In this way, the implantable prostheses may exhibit palpability and/or other properties akin to natural tissue. In some embodiments, the prostheses may be formed of materials which allow invasion of fibroblasts to produce collagen, which may wrap around the underlying material of the prosthesis. Accordingly, in some embodiments, the prostheses of the present disclosure may serve as a building block for in vivo organ development (engineering) or supplementation by providing a scaffold to induce vascularity.

In some embodiments, an implantable prosthesis may be formed of an assembly of conical mesh subunits or bodies connected to at least one neighboring subunit. The prosthesis may have an assembled shape akin to an ellipsoid. In some embodiments, an implantable prosthesis may be constructed by first forming each conical mesh subunit or body, as will be described in greater detail below, and subsequently connecting each conical mesh subunit to a neighboring subunit to form an ellipsoidal prosthesis. An ellipsoidal shape (e.g., spherical) may have the benefit of suitably fitting within a tissue resection site (e.g., biopsy site, lumpectomy site), which may help retain natural palpability of the tissue, such that the prosthesis or the resection site is not substantially palpable on the subject.

In some embodiments, an implantable prosthesis may be formed of an assembly of conical mesh subunits or bodies connected to at least one neighboring subunit. The prosthesis may be substantially mechanically isotropic. In some embodiments, an implantable prosthesis may be constructed by first forming each conical mesh subunit or body, as will be described in greater detail below, and subsequently connecting each conical mesh subunit to a neighboring subunit to form a substantially mechanically isotropic prosthesis. As will be described in greater detail below, substantially mechanical isotropic refers to the property of having similar compressive stiffnesses along more than one orientation of the prosthesis. A substantially mechanically isotropic implantable prosthesis may have the benefit of mimicking natural tissue during palpation, as well as providing uniform structural support to the anatomy. Accordingly, the prosthesis may exhibit mechanical properties commensurate with the natural tissue, such that the prosthesis or the resection site is not substantially palpable on the subject.

In some embodiments, an implantable prosthesis may be formed of an assembly of conical mesh subunits or bodies, each having a sidewall which may be connected to a sidewall of neighboring or adjacent subunit or body. Such a prosthesis may be constructed by first forming each conical mesh subunit or body, and connecting a sidewall of each body to a sidewall of an adjacent body. The connections between the various subunits or bodies may enhance the mechanical robustness of the prosthesis. The connections formed between the sidewalls of the bodies may serve to unify the conical subunits into the final implantable prosthesis such that pressure from the surrounding anatomy may be uniformly distributed within the prosthesis with a reduced risk of unraveling.

In some embodiments, an implantable prosthesis may be formed of an assembly of subunits or bodies each connected to another one of the subunits. The subunit may include a first portion connected to another portion of the subunit and arranged inside a volume defined by the other portion of the subunit. Such a prosthesis may be constructed by first forming each of the subunits, arranging a portion of the subunit to another portion of the subunit, connecting the two portions, and connecting each subunit to a neighboring or adjacent subunit. In this way, the subunits of the prosthesis may benefit from greater volume of material, which can further strengthen the prosthesis and support the natural tissue. As will be described in greater detail below, the portions of the subunit may differ in geometry to induce tissue ingrowth while optimizing the mechanical properties of the prosthesis.

In some embodiments, the prostheses of the present disclosure may induce tissue infiltration through porosity, which may enable cells to proliferate through the prostheses. The prosthesis may have porosity on multiple length scales. For example, the inner volume of the conical subunits of a prosthesis may provide large voids for tissue ingrowth. The subunits themselves may, be formed of a mesh-like or macroporous (having large pores) material, which may allow the prosthesis to accommodate sufficient autologous fat, biological materials, microphages, fibroblast, collagen, hyaluronic acid and or bioactive agents, in order to facilitate vascularization and tissue in-growth within the prosthesis. In some embodiments, the prostheses may be formed of materials with pores greater than 10 microns to limit the risk of rejection and formation of scar tissue. The term “macroporous” or “mesh” as used herein refers to an average pore size diameters of greater than or equal to 10 microns, 20 microns, 30 microns, 40 microns, 50 microns, 60 microns, 75 microns, 100 microns, and/or any other suitable pore size.

In some embodiments, the implantable prostheses of the present disclosure may preferably have a generally ellipsoidal shape to mimic the anatomical cavity left behind by a tissue removal procedure (e.g., lumpectomy), and/or any other natural or surgically formed cavity. Specifically, in some embodiments, the prostheses may have a spherical shape. However, it should be appreciated that the implantable prostheses of the present disclosure may have any suitable three-dimensional shape, including, but not limited to a sphere, ellipsoid, hemisphere, cylinder, cone, dome, cuboid, tetrahedron, triangular or square prism, dodecahedron, combinations thereof, and/or customized geometries. It should be appreciated that the term “ellipsoidal” as used herein refers to ellipsoidal three-dimensional shapes (which may have different average diameters in two or more directions), spheroidal shapes, and spherical shapes (which may have substantially similar average diameters in all directions).

It should be appreciated that the prostheses of the present disclosure may have any suitable size to accommodate a given application. For example, the prostheses may be sized to fit within a tissue removal (e.g., lumpectomy) site. Thus, the prosthesis may be any suitable size. The prosthesis may be characterized by an average diameter, which may preferably be between approximately 2 cm and 5 cm in some preferred embodiments, but other sizes are also contemplated, including prostheses with an average diameter of greater than or equal to 0.5 cm, 1 cm, 1.5 cm, 2 cm, 3 cm, 4 cm, 5 cm, 6 cm, 7 cm, 8 cm, and/or any other suitable size. The prostheses may also have an average diameter of less than or equal to 8 cm, 7 cm, 6 cm, 5 cm, 4 cm, 3 cm, 2 cm, 1.5 cm, 1 cm, 0.5 cm, and/or any other suitable size. Combinations of the foregoing, including prostheses having average diameters between 0.5 cm and 5 cm and between 2 cm and 8 cm are also contemplated, including prostheses larger than or smaller than the aforementioned ranges.

In some embodiments, a prosthesis may have a first average diameter across a first direction of the prosthesis and a second average diameter across a second direction. For example, the prosthesis may be generally ellipsoidal. Thus, it should be appreciated that the aforementioned ranges of average diameter may be employed relative to any suitable dimension of the prosthesis, as the present disclosure is not limited by the geometry of the implantable prosthesis.

As described previously, in some embodiments, implantable prostheses may be formed of twelve connected subunits. However, it should be appreciated that any suitable number of subunits may be employed to form any suitable shape of an implantable prosthesis. A prosthesis may have greater than or equal to 1, 5, 10, 15, 20, 25, 30, 35, 40, 50, and/or any other suitable number of subunits. A prosthesis may also have less than or equal to 50, 40, 35, 30, 25, 20, 15, 10, 5, 1, and/or any other suitable number of subunits. Combinations of the foregoing, including prostheses having between 1 and 50 and between 1 and 12 subunits, are also contemplated, along with number of subunits above the aforementioned ranges. As will be described in detail below, in some embodiments, multi-layer subunits may be employed for enhanced mechanical compressibility. Accordingly, the prostheses of the present disclosure are not limited by the number of constituent subunits.

In some embodiments, the subunits of the prostheses may also have a three-dimensional shape, such as the aforementioned shapes. In some embodiments, combinations of subunit geometries may be employed to achieve a suitable mechanical behavior. For example, a subunit of a prosthesis may have a generally conical shape.

It should be appreciated that the term “conical” or “cone” as used herein refers to both conventional conical and cone-like shapes, as well as partial conical shapes, such as a conical frustum shape, which may not have a sharp tip.

The subunits of the implantable prostheses described herein may be arranged and fixed in a three-dimensional configuration through any suitable means. In some embodiments, the subunits may be fixed in their three-dimensional configuration through any suitable bonding, thermal sealing, welding (e.g., ultrasonic or otherwise), adhesive bonding, combinations thereof, and/or any other suitable technique. In some embodiments, the subunits may be fixed in their three-dimensional configuration through a permanent or non-permanent arrangement. For example, fasteners, such as staples or sutures, may be used to non-permanently form the subunit and/or join neighboring subunits together. It should be appreciated that any of the aforementioned fixing techniques may be used to fix neighboring subunits together. Any suitable combination of fixing techniques to form the prosthesis may be employed, as the present disclosure is not so limited.

In some embodiments, as described in greater detail below relative to the figures, the subunits used in the implantable prostheses used herein may be fabricated from two-dimensional substrates. The substrates themselves may be formed from two-dimensional sheets. The two-dimensional substrates may be formed using any suitable technique, including, but not limited to, trimming or cutting with scissors, blades, other sharp cutting instruments, or thermal knives, laser-cutting techniques, welding techniques, die-cutting techniques, combinations thereof, and/or any other suitable technique. In other embodiments, the substrates may be formed using additive manufacturing techniques, such as 3D printing.

The implantable prostheses of the present disclosure may be formed of a material which may promote rapid tissue or muscle in-growth into and around the prosthesis. In some embodiments, the prosthesis may be formed from one or more layers of knitted mesh fabric. Non-limiting examples of surgical materials which may be utilized include BARD Mesh (available from C.R. Bard, Inc.), BARD Soft Mesh (available from C.R. Bard, Inc.), SOFT TISSUE PATCH (microporous ePTFE—available from W.L. Gore & Associates, Inc.); SURGIPRO (available from US Surgical, Inc.); TRELEX (available from Meadox Medical); PROLENE and MERSILENE (available from Ethicon, Inc.); PHASIX Mesh (available from C.R. Bard, Inc.), polyglactin (VICRYL—available from Ethicon, Inc.) and polyglycolic acid (DEXON—available from US Surgical, Inc.), collagen materials such as COOK SURGISIS, available from Cook Biomedical, Inc., combinations thereof, and/or any other mesh materials (e.g., available from Atrium Medical Corporation). The implantable material may be formed of planar mesh substrates. In some embodiments, the mesh material may be formed from multifilament yarns and that any suitable method, such as knitting, weaving, braiding, molding and the like, may be employed to form the mesh material.

In some embodiments, the prostheses may be formed of permanent materials, such as non-degradable thermoplastic polymers, including polymers and copolymers of ethylene and propylene, including ultra-high molecular weight polyethylene, ultra-high molecular weight polypropylene, nylon, polyesters such as poly(ethylene terephthalate), poly(tetrafluoroethylene), polyurethanes, poly (ether-urethanes), poly(methylmethacrylate), polyether ether ketone, polyolefins, and poly(ethylene oxide). In other embodiments, prostheses may be formed of degradable materials, including but not limited to, thermoplastic or polymeric degradable materials. Combinations of the foregoing are also contemplated. In some embodiments, the prosthesis may be formed of one or more absorbable polymers or copolymers, absorbable thermoplastic polymers and copolymers, and/or absorbable thermoplastic polyesters. The prostheses may be formed of polymers including, but not limited to, polymers of glycolic acid, lactic acid, 1,4-dioxanone, trimethylene carbonate, 3-hydroxy butyric acid, 4-hydroxybutyrate, e-caprolactone, including polyglycolic acid, polylactic acid, polydioxanone, polycaprolactone, copolymers of glycolic and lactic acids, such as VICRYL® polymer, MAXON® and MONOCRYL® polymers, and including poly(lactide-co-caprolactones); poly(orthoesters); poly anhydrides; poly(phosphazenes); polyhydroxyalkanoates; synthetically or biologically prepared polyesters; polycarbonates; tyrosine polycarbonates; polyamides (including synthetic and natural polyamides, polypeptides, and poly(amino acids)); polyesteramides; poly (alkylene alkylates); polyethers (such as polyethylene glycol, PEG, and polyethylene oxide, PEO); polyvinyl pyrrolidones or PVP; polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyalkylene oxalates; polyalkylene succinates; poly (maleic acids); silk (including recombinant silks and silk derivatives and analogs); chitin; chitosan; modified chitosan; biocompatible polysaccharides; hydrophilic or water soluble polymers, such as polyethylene glycol, (PEG) or polyvinyl pyrrolidone (PVP), with blocks of other biocompatible or biodegradable polymers, for example, poly(lactide), poly(lactide-co-glycolide, or polycaprolactone and copolymers thereof, including random copolymers and block copolymers thereof.

In some embodiments, prostheses may be formed of material blends of absorbable polymers including, but not limited to, polymers of glycolic acid, lactic acid, 1,4-dioxanone, trimethylene carbonate, 3-hydroxy butyric acid, 4-hydroxybutyrate, e-caprolactone, 1,4-butanediol, 1,3-propane diol, ethylene glycol, glutaric acid, malonic acid, oxalic acid, succinic aid, adipic acid, or copolymers thereof. In some embodiments, the prostheses may be formed of poly-4-hydroxybutyrate or a copolymer thereof.

In some embodiments, an implantable prosthesis may provide a means to deliver cells, stem cells, differentiated cells, fat cells, muscle cells, platelets, pedicles, vascular pedicles, tissue masses, extracellular adipose matrix proteins, gels, hydrogels, hyaluronic acid, collagen, bioactive agents, drugs, antibiotics, and other materials to the implant site. The cells and tissues, which may be delivered and/or coated or injected into the prostheses, may be autologous. The prostheses may be used for autologous fat transfer. The cells added, coated or injected on the prosthesis may include pancreatic islet cells, hepatic cells, and stem cells genetically altered to contain genes for treatment of patient illnesses. The prostheses may comprise bioactive agents to stimulate cell in-growth, including growth factors, cell adhesion factors, cellular differentiating factors, cellular recruiting factors, cell receptors, cell-binding factors, cell signaling molecules, such as cytokines, and molecules to promote cell migration, cell division, cell proliferation and extracellular matrix deposition. The prostheses may also be partially or entirely coated and/or contain agents to prevent tissue adhesion, or agents to prevent cell proliferation, particularly to delay cell invasion into the prostheses.

In some embodiments, the implantable prostheses may be loaded, filled, coated, or otherwise incorporated with bioactive agents. Bioactive agents may be included in the prostheses for a variety of reasons. For example, bioactive agents may be included in order to improve tissue in-growth into the implant, to improve tissue maturation, to provide for the delivery of an active agent, to improve wettability of the implant, to prevent infection, and to improve cell attachment. The bioactive agents may also be incorporated into the material composition of the substrate of the subunits.

The prostheses can contain active agents designed to stimulate cell in-growth, including growth factors, cell adhesion factors including cell adhesion polypeptides, cellular differentiating factors, cellular recruiting factors, cell receptors, cell-binding factors, cell signaling molecules, such as cytokines, and molecules to promote cell migration, cell division, cell proliferation and extracellular matrix deposition. Such active agents include fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insulin-like growth factor (IGF), hepatocyte growth factor (HGF), interleukin-1-B (IL-1 B), interleukin-8 (IL-8), and nerve growth factor (NGF), and combinations thereof. As used herein, the term “cell adhesion polypeptides” refers to compounds having at least two amino acids per molecule that are capable of binding cells via cell surface molecules. The cell adhesion polypeptides include any of the proteins of the extracellular matrix which are known to play a role in cell adhesion, including fibronectin, vitronectin, laminin, elastin, fibrinogen, collagen types I, II, and V, as well as synthetic peptides with similar cell adhesion properties. The cell adhesion polypeptides also include peptides derived from any of the aforementioned proteins, including fragments or sequences containing the binding domains.

In some embodiments, the implantable prostheses may be loaded, filled, coated, or otherwise incorporated with wetting agents designed to improve the wettability of the various surfaces of the prostheses to allow fluids to be easily adsorbed onto the prosthesis surfaces, and to promote cell attachment and or modify the water contact angle of the prosthesis surface. Examples of wetting agents include polymers of ethylene oxide and propylene oxide, such as polyethylene oxide, polypropylene oxide, or copolymers of these, such as PLURONICS®. Other suitable wetting agents may include surfactants or emulsifiers.

In some embodiments, the implantable prostheses may be loaded, filled, coated, or otherwise incorporated with gels, hydrogels or living hydrogel hybrids to further improve wetting properties and to promote cellular growth throughout the prosthesis. Hydrogel hybrids consist of living cells encapsulated in a biocompatible hydrogel like gelatin, methacrylated gelatin (GelMa), silk gels, and hyaluronic acid (HA) gels.

Other bioactive agents that can be incorporated in the prostheses may include antimicrobial agents, in particular antibiotics, disinfectants, oncological agents, anti-scarring agents, anti-inflammatory agents, anesthetics, small molecule drugs, anti-adhesion agents, inhibitors of cell proliferation, anti-angiogenic factors and pro-angiogenic factors, immunomodulatory agents, and blood clotting agents. The bioactive agents may be proteins such as collagen and antibodies, peptides, polysaccharides such as chitosan, alginate, hyaluronic acid and derivatives thereof, nucleic acid molecules, small molecular weight compounds such as steroids, inorganic materials such as hydroxyapatite and ceramics, or complex mixtures such as platelet rich plasma. Suitable antimicrobial agents include: bacitracin, biguanide, triclosan, gentamicin, minocycline, rifampin, vancomycin, cephalosporins, copper, zinc, silver, and gold. Nucleic acid molecules may include DNA, RNA, siRNA, miRNA, antisense or aptamers.

In some embodiments, the implantable prostheses may be loaded, filled, coated, or otherwise incorporated with allograft material and xenograft materials, including acellular dermal matrix material and small intestinal submucosa (SIS). In an embodiment, the prosthesis may contain a vascular pedicle or other tissue mass. In some embodiments, the prostheses may incorporate systems for the controlled release of the therapeutic or prophylactic agents.

In some embodiments, the implantable prostheses may be loaded, filled, coated, or otherwise incorporated with allograft or xenograft tissue and cells prior to implantation, during implantation, or after implantation, or any combination thereof. In some embodiments, the prostheses may be coated with autologous tissue and cells from the patient prior to implantation, during implantation, or after implantation, or any combination thereof. The autologous tissue and cells may include one or more of the following autologous fat, fat lipoaspirate, fat tissue, injectable fat, adipose tissue, adipose cells, fibroblast cells, and stem cells, including human adipose tissue-derived stem cells, also known as preadipocytes or adipose tissue-derived precursor cells, and fibroblast-like stem cells. In one embodiment, the prostheses may be coated with autologous tissue and cells as described herein, and may also further comprise a vascular pedicle or other tissue mass. As will be evident herein, the prostheses are designed to create not only a shape for the implant, such as a breast implant, but also a large surface area that can retain the autologous tissue and cells to encourage tissue in-growth.

In some embodiments, the prosthesis may be formed of an absorbable material (e.g., polymer or copolymer) that may be substantially resorbed after implantation within a 1 to 24-month timeframe, or 3 to 18-month timeframe, and retain some residual strength for at least 2 weeks to 6 months.

In some embodiments, the polymers and copolymers composition of the prostheses may have low moisture contents to ensure the prostheses can be produced with stiffnesses comparable to natural tissue, prolonged strength retention, and good shelf life. In some embodiments, the polymers and copolymers that are used to prepare the prostheses have a moisture content of less than 1,000 ppm (0.1 wt %), less than 500 ppm (0.05 wt %), less than 300 ppm (0.03 wt %), less than 100 ppm (0.01 wt %), and/or less than 50 ppm (0.005 wt %).

It should be appreciated that the compositions used to prepare the prostheses may have a low endotoxin content. In some embodiments, the endotoxin content may be low enough so that the prostheses produced from the polymer compositions have an endotoxin content of less than 20 endotoxin units per prosthesis as determined by the limulus amebocyte lysate (LAL) assay. For example, the polymeric compositions used to prepare the prosthesis may have an endotoxin content of <2.5 EU/g of polymer or copolymer. In another example, the P4HB polymer or copolymer, or PBS polymer of copolymer have an endotoxin content of <2.5 EU/g of polymer or copolymer.

In some embodiments, the prostheses of the present disclosure may include one or more markers for external detection of the prosthesis location. For example, a prosthesis may include a radiopaque marker (e.g., metallic staple), which may be visible and distinct over the nearby anatomy during x-ray imaging. The markers may be formed of any suitable extended use approved medical materials that may be medically imaged. Medical imaging means include, for example, radiographic imaging modalities (e.g., x-ray imaging), magnetic resonance imaging (MRI), ultrasonography, fluoroscopy, or computed tomography. The marker may therefore be formed of any non-absorbable, biocompatible materials, which refers to a material that does not cause any adverse reactions to a patient's health and that does not disintegrate over the lifetime of the patient. Non-absorbable, biocompatible materials include, but are not limited to, metal containing materials, polymer materials, ceramic materials, or composite materials that include metals, polymers, or combinations of metals and polymers. Suitable metals include, but are not limited to, gold, iridium, nickel, rhodium, silver, tantalum, titanium, stainless steel and alloys thereof, combinations thereof, and/or others. Suitable polymers include, but are not limited to, polyvinyl alcohol, polyurethanes, polyolefins, polyesters, polypropylenes, polyimides, polyetherimides, fluoropolymers, thermoplastic liquid polymers (LCP) such as, for example, Vectra® by Celanese, polyethylether ketones such as, for example, PEEK™ by Vitrex, polyamides, polycarbonates such as, for example, Makrolon® by Bayer Polymers, polysulfones, polyethersufones, polyphenylsulfones such as, for example, Radel® by Rowland Technologies, nylon, nylon copolymers, combinations thereof, and/or others. In some embodiments, the marker may include a shape-memory material, including, but not limited to, nitinol, titanium, or any shape-memory polymers.

In some embodiments, the prostheses of the present disclosure may include one or more Brachytherapy seeds which release low levels of radiation for therapy. The seeds may be physically attached to the prostheses and, in some cases, may be embedded in the assembly of conical mesh bodies. The number and location of Brachytherapy seeds can be customized for targeted localization of radiation therapy. For example, the protheses may include between six and thirty-six seeds. In some cases, the seeds are arranged in a pattern as directed by a radiation oncologist and/or nuclear medicine physician. In general, the seeds may comprise suitable radioactive agents (e.g., I-125, Ir-192, Pd-103) and may have a metallic outer shell (e.g., stainless steel, titanium-aluminum-vanadium). The Brachytherapy seeds may be radiopaque. In embodiments in which the implanted prostheses resorbs, the seeds can remain in the body. In some embodiments, the subunit substrate may include one or more visual and/or tactile fiducial markers to facilitate assembly. In some embodiments, the fiducial markers may indicate the location of a fixation spot (e.g., weld spot). The fiducial markers may be visually/optically or otherwise be apparent to an operator for assembly purposes. In some embodiments, the fiducial markers may be colored differently from the prosthesis substrate or otherwise distinct from the underlying prosthesis substrate. In some embodiments, the fiducial markers may be colored differently than the underlying substrate. In some embodiments, the prosthesis may include multiple fiducial markers colored with more than one color, to differentiate the fiducial markers from one another. For example, a first group of fiducial markers may be used to indicate fixation points within a single subunit substrate with a first color, and a second group of fiducial markers may be used to indicate fixation points between neighboring subunit substrates. It should be appreciated that any combination of fiducial marker types may be employed on any of the prosthesis subunits of the present disclosure.

In some embodiments, the prosthesis, itself, may be colored. For example, the coloring may be used to enhance visualization and/or provide visual queues to aid surgeons intraoperatively. The prosthesis may be colored in a variety of different ways. In some embodiments, a dye/pigment (e.g., medical grade colorant) may be incorporated (e.g., impregnated) into the prosthesis. In some embodiments, a colored coating may be applied to an outer surface of the prosthesis.

The implantable prostheses of the present disclosure may exhibit any suitable mechanical property to mimic the natural anatomy. In some embodiments, the prostheses may exhibit compressive stiffnesses to mimic the mechanics of the natural tissue while still providing support to the surrounding anatomy following implantation. As used herein and as will be expanded on in further detail below, the compressive stiffness is defined as the normalized compressive load applied to a subject divided by its relative strain during a compression test.

The prostheses of the present disclosure may have any suitable compressive stiffnesses, including, but not limited to greater than or equal to 0.5 psi, 1 psi, 1.5 psi, 1.8 psi, 2 psi, 3 psi, 4 psi, 5 psi, 6 psi, 6.8 psi, 7 psi, 8 psi, 9 psi, 10 psi, 12 psi, 15 psi, and/or any other suitable stiffness. The prostheses may also exhibit compressive stiffnesses less than or equal to 15 psi, 12 psi, 10 psi, 9 psi, 8 psi, 7 psi, 6.8 psi, 6 psi, 5 psi, 4 psi, 3 psi, 2 psi, 1.8 psi, 1.5 psi, 1 psi, 0.5 psi, and/or any other suitable stiffnesses. Combinations of the foregoing, including prostheses with compressive stiffnesses between 0.5 psi and 15 psi, and between 1.8 psi and 6.8 psi, are also contemplated, as well as compressive stiffnesses above and below the aforementioned ranges. It should be appreciated that the stiffness may be designed to mimic or support the tissue at the implantation site. Thus, the prosthesis may have any suitable stiffness above or below the aforementioned ranges.

It should be appreciated that in some embodiments, the implantable prostheses of the present disclosure may have a substantially mechanically isotropic, which may refer to the isotropic nature of the prosthesis's compressive stiffness (and/or any other mechanical property). The prosthesis may exhibit similar mechanical properties at multiple orientations. In some embodiments, a prosthesis having substantially isotropic compressive stiffness refers to a prosthesis having a first compressive stiffness along a first direction and a second compressive stiffness along a second direction, such that the magnitude of the first and second compressive stiffnesses are within 30% of one another, although first and second compressive stiffnesses within other ranges (e.g., within 25%, 15%, 10%, 5%) are also contemplated. The first and second directions may refer to major/minor axes of the structure (e.g., when the prosthesis is formed in an ellipsoidal shape), or may refer to any other suitable direction. For example, a prosthesis having substantially mechanically isotropic can refer to a prosthesis with a compressive stiffness of 3 psi in a horizontal direction and a compressive stiffness between 2.6 and 3.4 psi in the vertical direction. Thus, the first and second directions may be perpendicular to one another, although other arrangements are also contemplated.

In other embodiments, the mechanical properties of the implantable prostheses may be anisotropic (e.g., orientation-dependent). It should therefore be appreciated that one or more orientations of the implantable prosthesis may exhibit any of the aforementioned mechanical properties. It should also be appreciated that the mechanical properties of the prosthesis may be selected to mimic the natural tissue properties of the implant site. Therefore, dependent upon the implant site, the mechanical properties of the prosthesis may be any suitable value above or below the aforementioned ranges.

In some embodiments, the implantable prostheses of the present disclosure may be sufficiently compressible to pass through an incision smaller than the prosthesis. For example, an implantable prosthesis having an average diameter of 2 cm may be compressed up to 0.5 cm so that it may fit through an incision site of 1.5 cm. It should be appreciated that the compressibility of the prosthesis may be temporary, and that the prosthesis may return to within 10% of its original size following compression.

In some embodiments, the implantable prostheses of the present disclosure may include features to significantly reduce the risk of migration of the prostheses. For example, the markers may have an added benefit of generating friction within the implant site, to limit the migration of the prosthesis. In other embodiments, the prostheses may include other features to limit migration and/or re-orientation of the marker following implantation. It should be appreciated that in embodiments where the prosthesis is formed of a generally resorbable material, tissue infiltration through the prosthesis may relatively fix or engage the prosthesis with the nearby tissue. Accordingly, in some embodiments, the prostheses may reduce the risk of migration through material properties.

The implantable prostheses of the present disclosure may be used in any suitable application. In some embodiments, the prostheses may be implanted into soft tissue following a biopsy (and/or any other procedure, such as a lumpectomy) during treatment of cancers, such as breast, abdominal, liver, muscle, kidney, lung, and prostate cancer. In some embodiments, the prosthesis may be used in soft tissue reconstruction applications, such that it may serve as a breast implant, breast lift device, breast augmentation device, nipple implant, facial reconstruction device, buttock implant, malar augmentation device, cosmetic repair device, soft tissue regeneration device, hernia implant, hernia plug, wound healing device, tissue engineering scaffold, scaffold for a vascular pedicle or other tissue mass, guided tissue repair/regeneration device, bulking or filling device, void filler, device for treatment of vesicoureteral reflux, cell seeded device, drug delivery device, combinations thereof, and/or any other suitable application.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

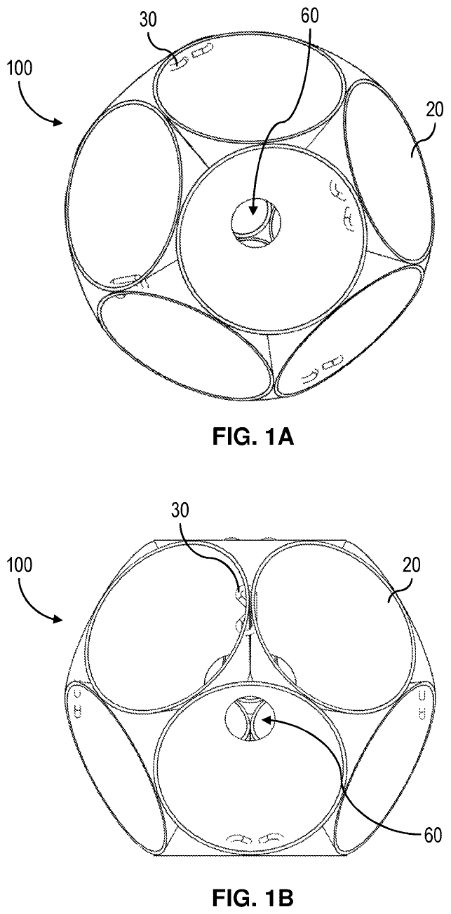

FIGS. 1A-1B show two isometric views of an implantable prosthesis 100 according to some embodiments. The prosthesis 100 may be formed of conical subunits 20, which may be arranged to form a generally ellipsoidal shape. Specifically, as shown in FIGS. 1A-1B, the prosthesis may have a generally spherical shape The central portion of the prosthesis 100 may include a hollow core 60 to allow for tissue in-growth, which may, in some embodiments, result in a more natural feel upon tissue infiltration of the implant. In some embodiments, the prosthesis may include one or more markers 30, which may have radiopaque properties. As described earlier, markers 30 may facilitate accurate visualization of the position of the prosthesis following implantation within the anatomy.

It should be appreciated that although prosthesis 100 shown in FIGS. 1A-1B is shown to have a generally spherical shape, prostheses of any suitable shape to fill a biopsy or lumpectomy cavity or serve other prosthesis purposes may also be employed. It should also be appreciated that although the prosthesis 100 shown in FIGS. 1A-1B is formed of an assembly of twelve conical subunits 20, any suitable number of subunits (conical or otherwise) may be employed to form any of the implantable prostheses described herein. Accordingly, the prostheses of the present disclosure are not limited by shape, size, number of subunits, shape of subunits, arrangement of subunits, and/or any other factor.

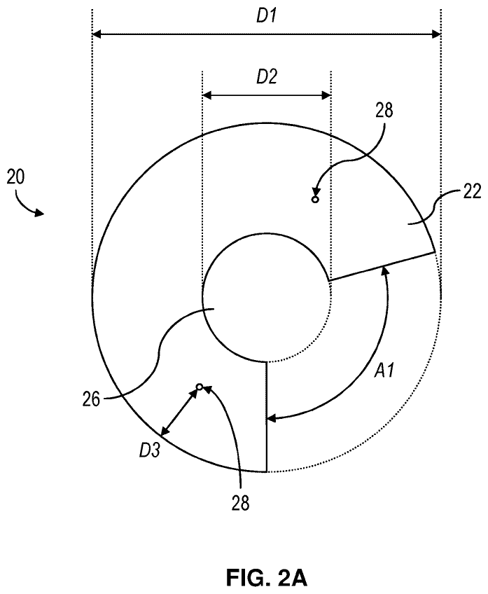

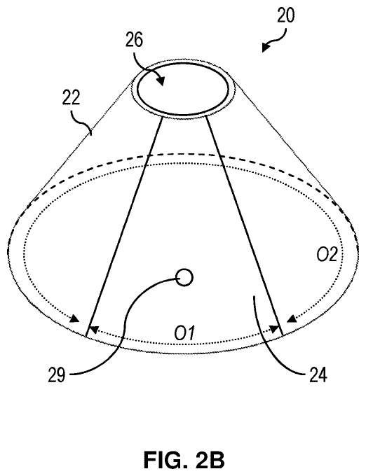

FIGS. 2A-2B show various views of a conical subunit 20 according to some embodiments. FIG. 2A depicts a top view of a substantially two-dimensional substrate 22, which can be manipulated (e.g., rolled) to form the conical subunit 20 shown in the isometric view of FIG. 2B. In some embodiments, the substrate can be formed of a porous biocompatible mesh material. In some embodiments, the end portions of the substrate 22 may be overlapped together to form an overlapping region 24, where the end portions may subsequently be fixed to one another. In some embodiments, a weld spot 29 may fix the end portions of the substrate 22 together, as shown in FIG. 2B, although other methods of fixation, both temporary and permanent, are also contemplated. This manipulation process may transform the substrate 22 from a substantially two-dimensional arrangement, as shown in FIG. 2A, to a three-dimensional arrangement with sidewalls, as shown in FIG. 2B. In some embodiments, the subunit may have a conical frustum (or “conical”) shape, whereas in other embodiments, the subunit may form a different three-dimensional shape.

The overlapping region 24 of each conical subunit 20, as shown in FIG. 2B, may contribute to the overall mechanical properties of the subunit and the prosthesis. For example, a large overlapping region may yield a stiffer subunit when compared to a smaller overlapping region. In part, this increase in stiffness may be due to the change in thickness of the subunit, being two layers rather than one layer. The overlapping region 24 may be defined by a degree of overlap O1, as shown in FIG. 2B, which may be added to a degree of non-overlap O2 to add up to approximately 360°. The degree of overlap O1 may be any suitable value to achieve a desired stiffness of the subunit. The degree of overlap may be greater than or equal to 5°, 10°, 15°, 20°, 25°, 30°, 35° 40°, 45° 50°, 55° 60°, 65°, 70°, 75° 80°, 85°, 900, 100°, and/or less than or equal to 100°, 95°, 90°, 85°, 80°, 75° 70°, 65°, 60°, 55° 50°, 45°, 40°, 35°, 30°, 25°, 20°, 15°, 10°, 5°, and/or combinations thereof.

It should be appreciated that although the fixation points (e.g., weld spots) 29 are depicted to be substantially circular, non-circular fixation points are also contemplated. In some embodiments, an oblong or elongated fixation point may be employed to produce an enhanced fixation between the substrate portions and/or between neighboring subunits. In some embodiments, an oblong or elongated fixation point may help fix the various elements in more than one direction. In some embodiments, an oblong or elongated fixation point may replace multiple circular fixation points. For example, a series of three weld spots may be replaced by one elongated fixation point. Such a replacement may expedite assembly processes. In some embodiments, the elongated fixation point may enhance the stiffness of the subunit.

In some embodiments, as shown in FIG. 2A, the substrate 22 may be formed in a generally c-shaped arrangement. A central portion 26 of the substrate 22 may be removed to enable tissue in-growth through the implantable prosthesis, as shown in FIGS. 1A-1B. The substrate 22 may also include a cutout spanning a cutout angle A1 around the substrate 22, as shown in FIG. 2A. Such a cutout may enable the substrate 22 to be manipulated to form the sidewalls of a three-dimensional cone. In some exemplary embodiments, the subunit 20, shown in FIG. 2B, may have a sidewall angle of approximately 63°, such that twelve identical subunits together may enable the formation of a generally spherical implantable prosthesis. In some embodiments, the sidewall angle of the subunits may be above or below 63°, including between 50° and 70°, between 60° and 65°, and/or any other suitable range of sidewall angles. Of course, prostheses employing different numbers of subunits with different geometries, in order to form spherical or non-spherical prostheses, are also contemplated.

The cutout angle A1 shown in FIG. 2A may be any suitable angle to enable the formation of a conical frustum. In some embodiments, the substrate may include a cut, rather than a cutout, spanning an angle A1 approximately equal to 0°. In some embodiments, the cutout angle A1 may be greater than or equal to 0°, 5°, 10°, 15°, 20°, 30°, 40°, 50°, 65°, 80°, 90°, 100°, 120°, 135°, 150°, 180°, and/or any other suitable angle. The cutout angle A1 may also be less than or equal to 180°, 150°, 135°, 120°, 100°, 90°, 80°, 65°, 50°, 40°, 30°, 20°, 15°, 10°, 5°, 0°, and/or any other suitable angle. Combinations of the foregoing, including cutout angles between 0° and 180°, are also contemplated. In some embodiments, the cutout angle A1 may be 40°. In other embodiments, the cutout angle A1 may be 65°. In other embodiments still, the cutout angle A1 may be 135°. Of course, cutout angles above and below the aforementioned ranges are also contemplated. It should be appreciated that any of the implantable prostheses of the present disclosure may be formed of more than one conical (or otherwise) subunits having the same or different cutout angles.

In some embodiments, the substrate 22 may be characterized by an average diameter D1. The average substrate diameter D1 may be any suitable size to suitably fit within the implant site and/or accommodate any other suitable application. In some exemplary embodiments, the average diameter D1 of the substrate 22 may be greater than or equal to 1 cm, 1.5 cm, 2 cm, 2.2 cm, 2.5 cm, 2.8 cm, 3 cm, 3.2 cm, 3.5 cm, 3.8 cm, 4 cm, 4.5 cm, 5 cm, 6 cm, 7 cm, 8 cm, and/or any other suitable size. The average diameter D1 of the substrate 22 may also be less than or equal to 8 cm, 7 cm, 6 cm, 5 cm, 4.5 cm, 4 cm, 3.8 cm, 3.5 cm, 3.2 cm, 3 cm, 2.8 cm, 2.5 cm, 2.2 cm, 2 cm, 1.5 cm, 1 cm, and/or nay other suitable size. Combinations of the foregoing ranges, including average substrate diameters between 1 cm and 8 cm, are also contemplated, as well as sizes above and below the aforementioned ranges.

In some embodiments, the central portion 26 of the substrate 22 may be characterized with a core percentage value, representing the ratio of the average diameter D2 of the central portion 26 to an average diameter D1 of the substrate 22. The core percentage may be any suitable value to enable sufficient tissue in-growth in the implantable prosthesis, while still maintaining sufficient mechanical stiffnesses to support the surrounding tissue following implantation. The core percentage may be any suitable value, greater than or equal to 5%, 10%, 15%, 20%, 25%, 30%, 35%, 40%, 50%, and/or any other percentage. The core percentage may also be less than or equal to 50%, 40%, 35%, 30%, 25%, 20%, 15%, 10%, 5%, and/or any other suitable percentage. Combinations of the foregoing ranges, including core percentages between 10% and 35% and between 5% and 50%, are also contemplated, as well as ranges above and the aforementioned ranges.

In some embodiments, the core percentage of the subunits may result in a total hollow core percentage of the implantable prosthesis, which may represent the ratio between the hollow central volume of the prosthesis and the total volume of the prosthesis. The hollow core percentage of the prosthesis may be greater than, equal to, or less than the core percentage of any given subunit of the prosthesis.

In some embodiments, the substrate 22 may include one or more fiducial markers 28, shown in FIG. 2A, to facilitate the assembly of the conical subunit, shown in FIG. 2B. The end portions of the substrate 22 may be folded together to align the fiducial markers 28 and subsequently fixed together (e.g., using a welding technique) to form the sidewalls of the three-dimensional subunit. Of course, embodiments without fiducial markers are also contemplated.

In some embodiments, the fiducial marker and subsequently, the weld (or other fixing technique) may be spaced away from the outer edge by a distance D3, as shown in FIG. 2A. This distance may be sufficiently sized to enable a practitioner to secure the implantable prosthesis to tissue with a fastener. The weld may therefore be spaced away from the edge to provide clearance for the fastener. In some embodiments, the distance may also provide ample space for other fixing processes, such as the fixation between neighboring subunits. The distance D3 may be any suitable value greater than or equal to 2 mm, 2.5 mm, 3 mm, 5 mm, and/or any other suitable distance away from the edge of the substrate. The distance may also be less than or equal to 5 mm, 3 mm, 25 mm, 2 mm, and/or any other suitable distance away from the edge of the substrate. It should be appreciated that although a single fiducial marker 28 and weld spot 29 is shown in FIGS. 2A-2B, subunits having more than one fiducial marker and weld spots are also contemplated. In some embodiments, two weld spots may enhance the stiffness of the subunit.

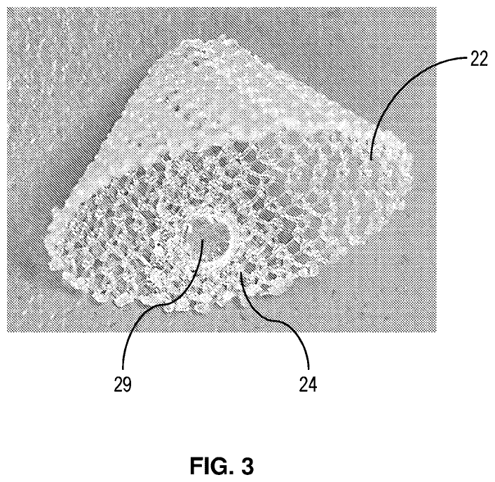

FIG. 3 depicts a conical subunit according to some embodiments. As shown, the substrate 22 of the subunit may be overlapped within a region 24 to enable the three-dimensional configuration. FIG. 3 also depicts a weld spot 29 formed in the overlapping region 24. As shown, the weld spot 29 may physically and permanently alter the substrate 22 in order to fix the subunit in its three-dimensional configuration. However, embodiments in which the subunit is temporarily arranged in its three-dimensional configuration, using for example, fasteners such as staples, are also contemplated.

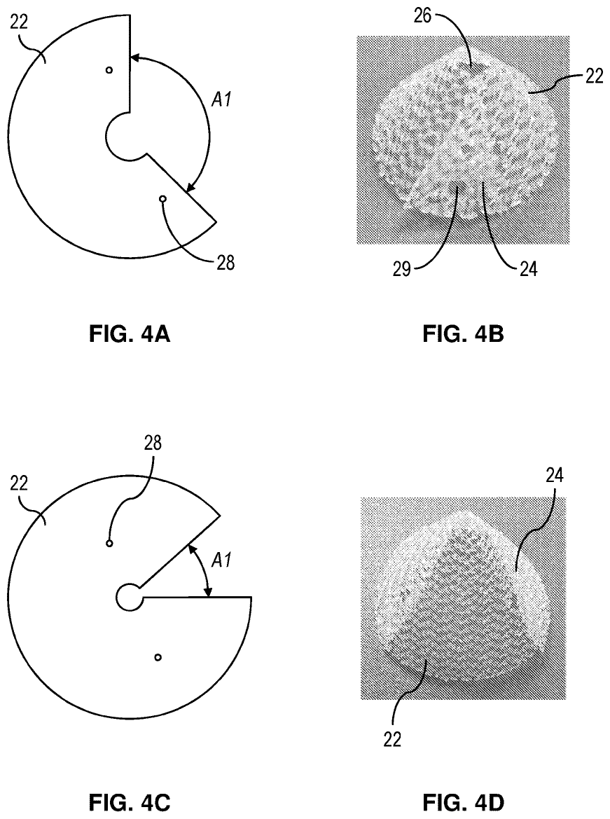

FIGS. 4A-4D depict various embodiments of conical subunits having different cutout angles A1. As shown, in some embodiments, the cutout angle may determine the extent of the overlapping region 24. FIGS. 4B and 4D show subunits having similar sidewall angles. However, given that the cutout angle A1 of the subunit of FIG. 4B (see FIG. 4A) is significantly larger than the cutout angle A1 of the subunit of FIG. 4D (see FIG. 4C), the overlapping region 24 of the subunit from FIG. 4D is significantly larger. In some embodiments, an extended overlapping region may result in enhanced stiffness. It should be appreciated that dependent upon the final desired sidewall angle of the subunit, the overlapping region extension may be selected irrespective of the cutout angle.

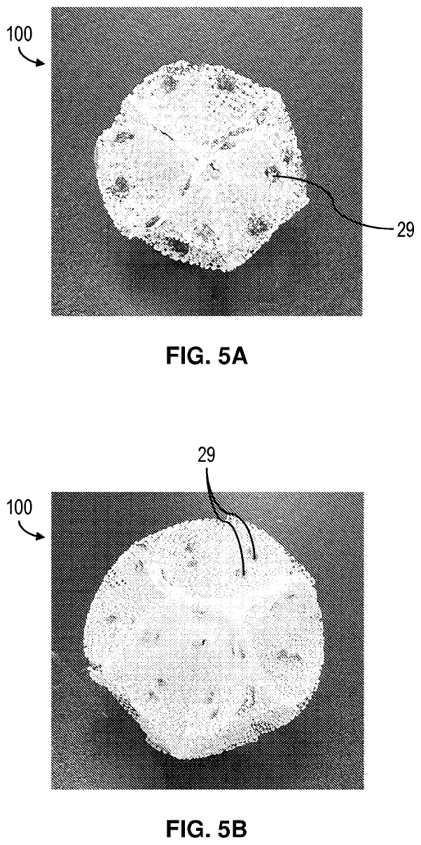

FIGS. 5A-5B depict two exemplary embodiments of implantable prostheses 100. Both embodiments include twelve conical subunits formed in a generally spherical arrangement. In some embodiments, as exemplified by FIG. 5A, each cone may be fixed to a neighboring cone through one weld spot 29. In other embodiments, as exemplified by FIG. 5B, each cone may be fixed to a neighboring cone through two weld spots 29. In some embodiments, increased weld spots (between cones or within a single cone) may increase the overall stiffness of the implantable prosthesis.

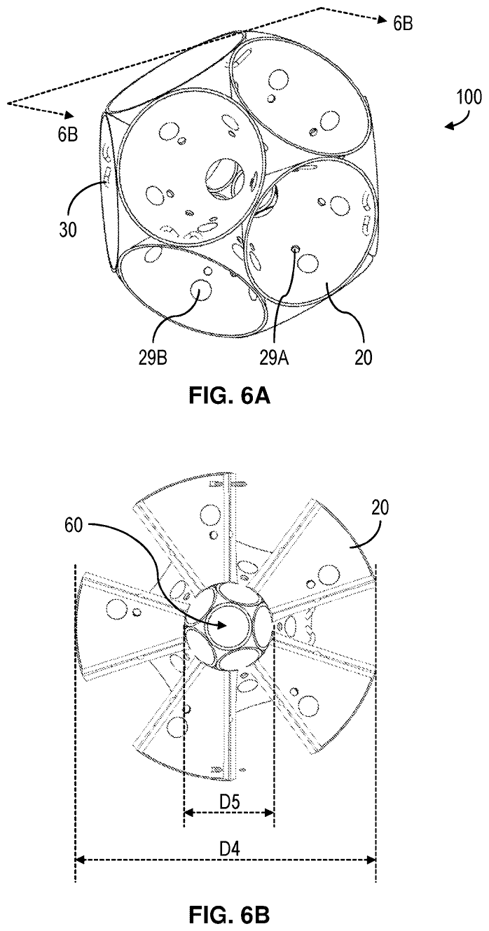

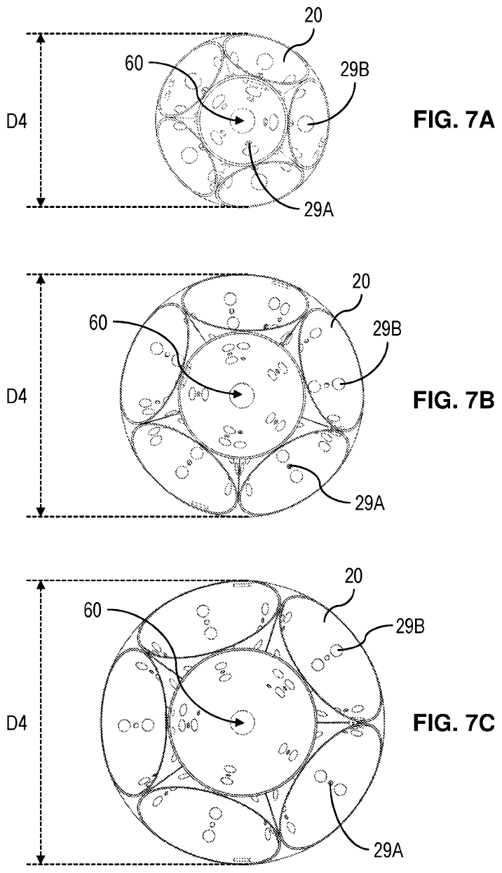

FIGS. 6A-6B depict an implantable prosthesis 100 according to some embodiments. The prosthesis 100 may be formed of twelve subunits 20, which have a generally conical frustum shape, as shown in FIG. 6A. Each subunit 20 may include one or more weld spots 29A from the formation of the subunit itself, and one or more weld spots 29B from the assembly of the prosthesis. In other words, weld spots 29A may be applied in intra-subunit formation, and weld spots 29B may be applied in inter-subunit assembly to fix neighboring subunits to one another. The prosthesis may also include one or more markers 30 to facilitate external visualization of the prosthesis using various medical imaging modalities (e.g., x-ray, MRI).

FIG. 6B depicts a cross-section of the prosthesis 100 of FIG. 6A taken along line 6B-6B. As shown, the core 60 of the prosthesis may have an average core diameter D5, which may be formed as a result of the frustum shape of the subunits 20. As described in greater detail above, the prosthesis may have a hollow core percentage proportional to the ratio of the average core diameter D5 to the average prosthesis diameter D4, as shown in FIG. 6B. The average prosthesis diameter D4 of FIG. 6B may be approximately 3 cm, but diameters larger and smaller than 3 cm are also contemplated, as described in greater detail above.

The average core diameter D5 may be any suitable value to induce tissue in-growth while maintaining suitable mechanical stiffness to support the nearby tissue. The average core diameter D5 may be greater than or equal to 0.05 cm, 0.1 cm, 0.2 cm, 0.5 cm, 1 cm, 2 cm, and/or any other suitable size. The average core diameter D5 may also be less than or equal to 2 cm, 1 cm, 0.5 cm, 0.2 cm, 0.1 cm, 0.05 cm, and/or any other suitable size. Combinations of the foregoing ranges, including average core diameters D5 between 0.05 cm and 2 cm, are also contemplated, as well as diameters above and below the aforementioned ranges. The average core diameter D5 may also be any suitable percentage of the average prosthesis diameter D4. In some embodiments, the average core diameter D5 may be greater than or equal to 2%, 5%, 10%, 15%, 20%, 25%, 30%, 35%, 40%, 50%, 60%, 75%, and/or any other percentage of the average prosthesis diameter. The average core diameter D5 may also be less than or equal to 75%, 60%, 50%, 40%, 35%, 30%, 25%, 20%, 15%, 10%, 5%, 2%, and/or any other suitable percentage of the average prosthesis diameter. Combinations of the foregoing ranges, including average core diameters D5 between 10% and 35% and between 2% and 75% of the average prosthesis diameter, are also contemplated, as well as ranges above and the aforementioned ranges. It should be appreciated that any suitable size of the average core and prosthesis diameters (and any suitable ratio thereof) may be employed, as the present disclosure is not limited by the geometry of the core.

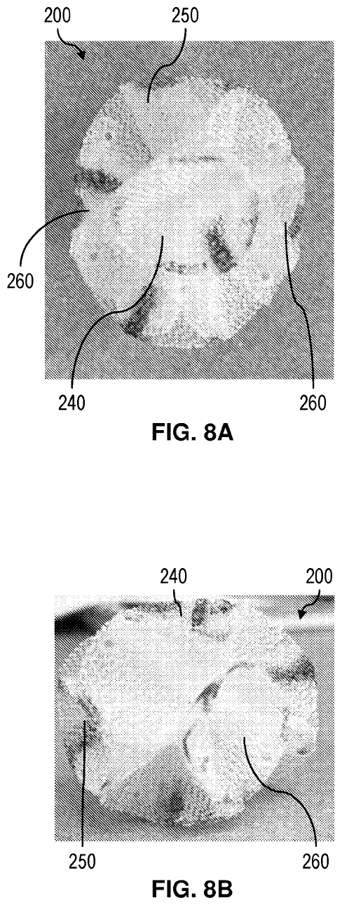

FIGS. 7A-7C show three embodiments of spherical prosthesis formed of twelve conical subunits 20 each. All three prostheses have similarly sized cores 60 (e.g., similar core diameters). However, the three prostheses differ in their average diameter. The prosthesis of FIG. 7A has an average diameter D4 of approximately 2 cm, FIG. 7B has an average diameter D4 of approximately 4 cm, and FIG. 7C has an average diameter D4 of approximately 5 cm. Thus, the hollow core percentage of the prosthesis shown in FIG. 7C may be smaller than the hollow core percentage of the prosthesis shown in FIG. 7A.

In some embodiments, the number of fixation points (e.g., markers and weld spots) may depend on the size of the prosthesis. For example, FIG. 7A shows a prosthesis where each subunit 20 has 5 intra-subunit weld spots 29A to enhance the stiffness of each subunit, and 5 inter-subunit weld spots 29B, where the subunit is fixed to each of its neighboring subunits once. FIGS. 7B-7C both show prostheses where each of their twelve subunits include 6 intra-subunit weld spots 29A and twelve inter-subunit weld spots 29B. However, the arrangement and absolute positions of the weld spots of FIGS. 7B and 7C differ due to the difference in the average prosthesis diameter D4.

It should be appreciated that prostheses having different subunits, each having a different number and/or arrangement of subunits, is also contemplated. It should also be appreciated that any of the implantable prostheses of the present disclosure may be formed of any combination of subunits. In some embodiments, the prosthesis may be formed of a number of similar subunits, as shown in FIGS. 5A-7C. In other embodiments, the prosthesis may be formed of different subunits. For example, an implantable prosthesis may include a first group of subunits having a first core percentage, a first average substrate diameter, and a first cutout angle, and a second group of subunits having a second core percentage, a second average substrate diameter, and a second cutout angle. Thus, it should be appreciated that the implantable prostheses of the present disclosure may employ any number of subunits to form any combination of geometric, structural, and/or mechanical properties.

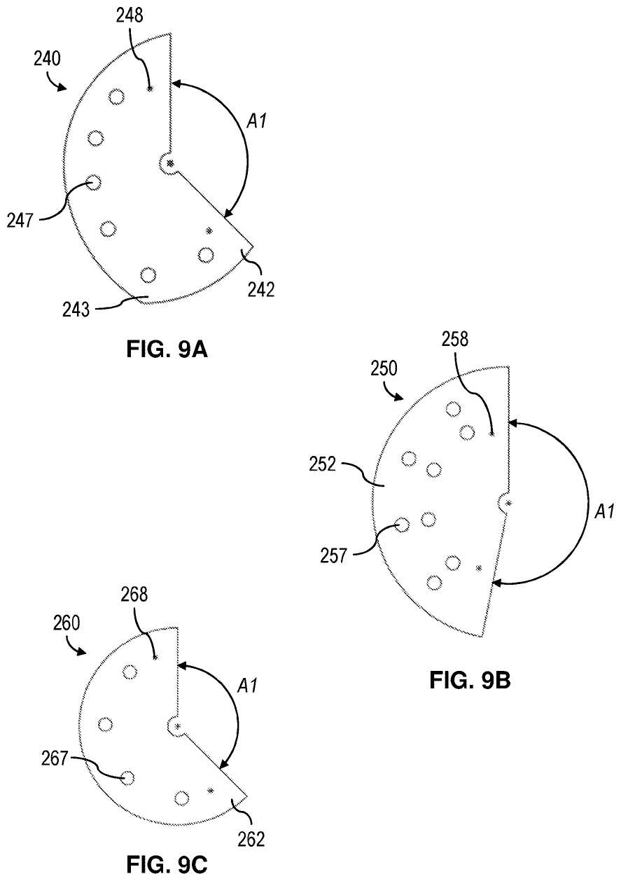

FIGS. 8A-8B depict various views of an implantable prosthesis 200 having a generally ellipsoidal shape. In some embodiments, the ellipsoidal shape of the prosthesis 200 may be achieved through a combination of three types of subunits: side cones 240, peak cones 250, and middle cones 260. The variation of subunit type enables an implantable prosthesis with an elongated ellipse to accommodate similarly-shaped implant sites (e.g., biopsy or lumpectomy sites).

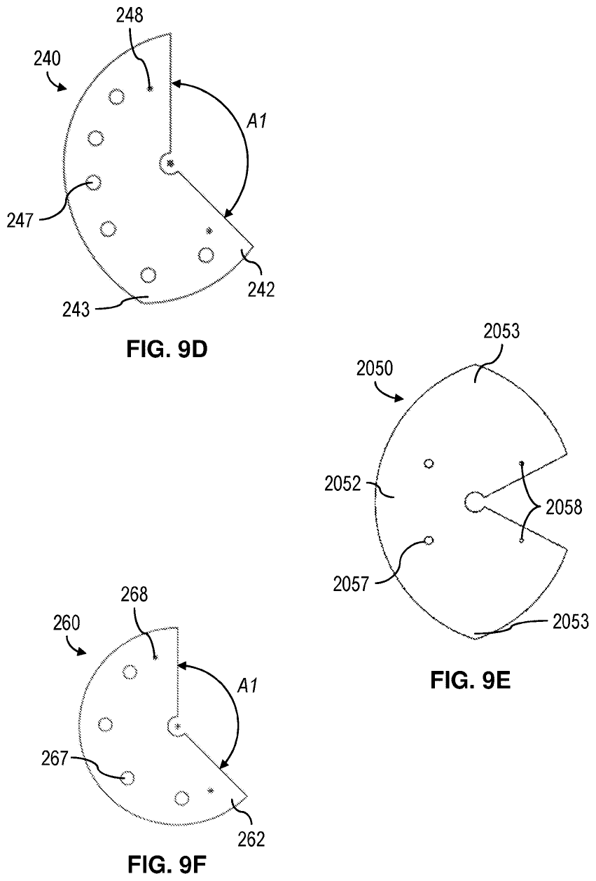

FIGS. 9A-9F show substrate geometries for the three subunits that form the prosthesis 200 of FIGS. 8A-8B. In some embodiments, the side cone 240 may include a generally c-shaped substrate with a cutout, which can be characterized by a cutout angle A1. It should be appreciated that the side cone substrate 242 may include an extended portion 243, which may partially skew the c-shape of the side cone 240. The variation in geometry enables the side cone to form a rounded ellipsoidal shape prosthesis, when used along with the peak and middle cones. Similar to the embodiments described earlier in relation to FIG. 2A, the side cone 240 may include one or more fiducial markers 248 to indicate intra-subunit weld points, as well as one or more fiducial markers 247 for cone-cone (or inter-subunit) weld points. As described previously, any number of weld points for the subunit and/or between the subunit and its neighboring subunits may be employed to achieve desired mechanical properties. In some embodiments, no fiducial markers may be employed.

FIG. 9B depicts a peak cone and FIG. 9C depicts a middle cone 260 according to some embodiments. Similar to the embodiment shown in FIG. 2A, the peak and middle cones may include substrates 252, 262, along which cutouts may be formed. The peak cone cutout angle A1 may be larger than the middle cone cutout angle A1 to enable the formation of a differently sized three-dimensional cone. For example, the peak cone cutout angle A1 may be 191.25° as shown in FIG. 9B. However, other peak cone cutout angles are also contemplated, including between 100°-250° and between 135°-210°. It should be appreciated that the peak cone cutout angle may vary depending on the size of the implantable prosthesis. Accordingly, any suitable peak cone cutout angle may be employed. FIG. 9C shows an exemplary middle cone cutout angle A1 of 135°, but as noted relative to the peak cone cutout angle, any suitable cutout angle may be employed, such as between 40°-140°. Of course, the cutout angles of any of the subunits of the present disclosure may be any suitable magnitude to enable the formation of sidewalls for a suitable three-dimensional subunit. Both peak and middles cones may include one or more fiducial markers 258, 268 to indicate intra-subunit weld points, as well as one or more fiducial markers 257, 267 for cone-cone (or inter-subunit) weld points. As described previously, any number of weld points for the subunit and/or between the subunit and its neighboring subunits may be employed to achieve desired mechanical properties. In some embodiments, no fiducial markers may be employed.

As described previously, in some embodiments, an implantable prosthesis may be ellipsoidal and non-spherical. In some embodiments, the ellipsoidal prosthesis may have an average height and an average width. Table 1 below summarizes an exemplary list of geometric properties of ellipsoidal implantable prostheses. Each of the prostheses listed in table 1 is formed of fourteen total conical subunits, which include 2 peak cones, 8 side cones, and 4 middle cones, as previously described.

TABLE 1
tabulated exemplary geometric properties
of ellipsoidal, non-spherical prostheses.

| Ellipsoidal Prosthesis | Cone | Hollow Core | Overlap (Degrees |
|---|---|---|---|
| Size (Width × Height) | Type | Percentage (%) | of Circle Removed) (°) |
| 2 cm × 3 cm | Peak | 25 | 200 |
| | Side | 20 | 120 |
| | Middle | 25 | 125 |
| 3 cm × 4 cm | Peak | 24.5 | 218 |
| | Side | 13 | 120 |
| | Middle | 10 | 120 |
| 4 cm × 5 cm | Peak | 10 | 120 |
| | Side | 10 | 55 |
| | Middle | 10 | 35 |

In some embodiments, an ellipsoidal prosthesis may utilize peak, side, and middle cones as shown and described relative to FIGS. 9A-9C. In other embodiments, an ellipsoidal prosthesis may employ a peak cone which may be different from cone 250 shown in FIG. 9B. For example, an ellipsoidal prosthesis may be formed of three conical subunits, including a side cone 240, as shown in FIG. 9D, which may be similar to the side cone 240 of FIG. 9A, a middle cone 260, as shown in FIG. 9F, which may be similar to the middle cone 260 of FIG. 9C, and a peak cone 2050, as shown in FIG. 9E, which may be different from peak cone 250 of FIG. 9B. Specifically, peak cone 2050 may be formed of a substrate 2052 having two extended portions 2053 which may partially skew the c-shaped formation of the peak cone 2050. Peak cone 2050 may also include one or more fiducial markers 2058 to indicate intra-subunit weld points, as well as one or more fiducial markers 2057 for cone-cone (or inter-subunit) weld points. As described previously, any number of weld points for the subunit and/or between the subunit and its neighboring subunits may be employed to achieve desired mechanical properties. In some embodiments, no fiducial markers may be employed.

It should be appreciated that the peak cone of FIG. 9E may be employed with ellipsoidal prostheses having a height of 5 cm and an average width of 4 cm to accommodate the larger geometry of the prostheses.

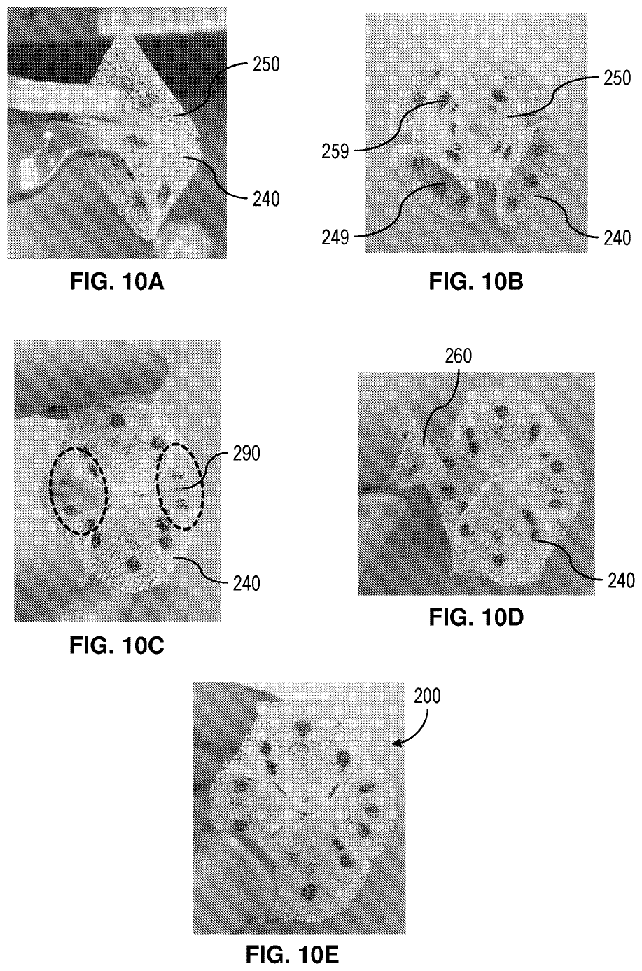

FIGS. 10A-10E depict a process for assembling an ellipsoidal implantable prosthesis, such as the one shown in FIGS. 8A-8B. The exemplary prosthesis may be formed of two peak cones, eight side cones, and four middle cones. However, it should be appreciated that any suitable number of any subunit may be employed. Initially, each subunit may be assembled from its two-dimensional substrate to a three-dimensional configuration, as previously described. Subsequently, as shown in FIG. 10A, a peak cone 250 may be aligned with a side cone 240 such that the top edges are aligned. The sidewalls of the two cones may be clamped together for alignment and subsequently fixed to one another (e.g., through a weld). Three other side cones 240 may then be fixed to the central peak cone 250, aided by fiducial markers 249, 259 on the side and peak cones, respectively, as shown in FIG. 10B. It should be appreciated that each side cone may be welded one or more times to the peak cone and additionally welded one or more times to each of its neighboring cones.

The subassembly of the peak cone 250 and four side cones 240 may be repeated to form two partial halves of the prosthesis. When the partial halves are aligned, as shown in FIG. 10C, four side cones from each partial half may be fixed to one another, connecting the two partial halves. However, as shown in FIG. 10C, there may be gaps 290 between the side cones in the central portion of the prosthesis. These gaps may be filled with middle cones 260, as shown in FIG. 10D. Each middle cone 260 may be welded at least once to four neighboring side cones 240. The final prosthesis formed of fourteen total subunits may look generally ellipsoidal, as shown in FIG. 10E. It should be appreciated that the assembly processes described in relation to FIGS. 10A-10E is non-limiting, and that any other assembly process may be employed to form the implantable prostheses of the present disclosure.

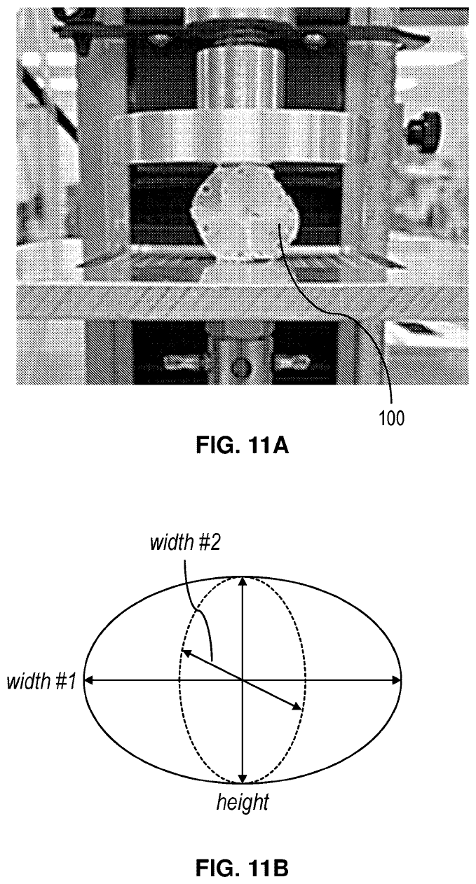

As described in greater detail above, the implantable prostheses may exhibit substantially isotropic mechanical properties such as compressive stiffness. In some embodiments, the implantable prostheses of the present disclosure may undergo compression testing to evaluate the stiffness and palpability of the prostheses, and determine the compressive stiffness of the prostheses. The testing may be done by compressing the prostheses up to 30% at a rate of 0.2 mm/s, which may be slow enough to achieve a quasi-static test condition. To evaluate a prosthesis, the prosthesis 100 may first be measured and subsequently positioned within a compression testing system (e.g., Instron), as shown in FIG. 11A. In some non-limiting embodiments, a 100 N load cell may be used to compress the prosthesis. The system may then compress the prosthesis up to 30% of its original height (e.g., a point of interest) and collect force displacement data.

The various geometric measurements of the prosthesis, such as the pre-compression height, displacement, and force may be used to calculate the compressive stiffness of the prosthesis. Specifically, the compressive stiffness of the prosthesis may be derived as follows:

$$\begin{matrix}Compressive Stiffness ( psi ) = \frac{Normalized Force ( \frac{lbf}{{in}^{2}} )}{Compressive Displacement ( mm )} \\ = \frac{\frac{Force Measurement ( lbf )}{Cross Sectional Area ( {in}^{2} )}}{\frac{Displacement ( mm )}{Prosthesis Height ( mm )}}\end{matrix}$$

In the equation above, the normalized force and compressive displacement are measured through the testing, and the prosthesis dimensions are measured prior testing. It should be appreciated that in some embodiments, given the non-linear slope of the compression curve, the term “compressive stiffness” as used herein refers to a compressive secant stiffness, defined by the linear slope between the origin and the point of interest.

FIG. 11B shows the various dimensions as they relate to an implantable prosthesis. The cross sectional area of the prosthesis can be calculated as follows:

$$Cross Sectional Area ( {in}^{2} ) = π * \frac{Prosthesis width \#1 ( mm )}{2 * 25.4} * \frac{Prosthesis width \#2 ( mm )}{2 * 25.4}$$

The force measurement from the testing system may be converted to a normalized force as follows:

$$Normalized Force ( \frac{lbf}{{in}^{2}} ) = \frac{Force Measurement ( lbf )}{Cross Sectional Area ( {in}^{2} )}$$

In one exemplary embodiment, an ellipsoidal implantable prosthesis with overall dimensions of approximately 2 cm by 2 cm by 3 cm, having a 25% hollow core percentage, may exhibit an average compressive stiffness of approximately 4.05 psi in the horizontal orientation and an average compressive stiffness of 4.66 psi in the vertical direction. In another exemplary embodiment, an ellipsoidal implantable prosthesis with overall dimensions of approximately 3 cm by 3 cm by 4 cm, having a 10% hollow core percentage, may exhibit an average compressive stiffness of approximately 3.55 psi in the horizontal orientation and an average compressive stiffness of 3.52 psi in the vertical direction. In yet another exemplary embodiment, an ellipsoidal implantable prosthesis with overall dimensions of approximately 4 cm by 4 cm by 5 cm, having a 10% hollow core percentage, may exhibit an average compressive stiffness of approximately 2.40 psi in the horizontal orientation and an average compressive stiffness of 2.82 psi in the vertical direction.

The stiffness of the implantable prostheses of the present disclosure may be adjusted through a variety of means, including, but not limited to, the size of the prosthesis, material composition of the prosthesis, type and number of fixation sites, hollow core percentage, among many others. In some embodiments, the stiffness of the implantable prosthesis may be increased by increasing the wall thickness of the subunits. The wall thickness may be increased through the use of thicker substrate material (e.g., mesh-like repair fabric). In some embodiments, the wall thickness of the subunits may be increased through the use of multiple overlapping subunits.

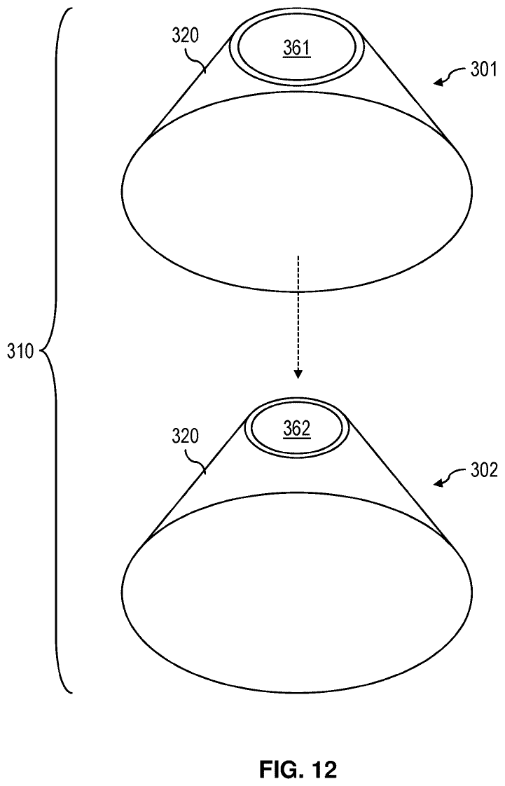

FIG. 12 depicts an exemplary partial assembly process for a double cone subunit 310. The subunit 310 may include a first conical subunit 301, similar to those described relative to FIGS. 2A-2B, having a first central portion 361, connected to a second conical subunit 302 with a second central portion 362. In some embodiments, the second conical subunit may be arranged inside a volume defined by the first conical subunit, such that they may overlap. In some embodiments, as shown in FIG. 12, the central portions of the first and second conical subunits may differ (e.g., central portion 362 may be smaller than central portion 361). In this way, the core of the implantable prosthesis formed of such a subunit 310 may have less material, which may induce a greater rate of tissue infiltration to enhance the repair process, while maintaining a compressive stiffness comparable to natural tissue to support the nearby anatomy. Of course, double cone subunits having two substantially similar conical subunits (e.g., similar central portions), are also contemplated.

It should be appreciated that any of the implantable prostheses of the present disclosure may employ a double layer subunit. In some embodiments, dependent upon the application, the prosthesis subunit may employ more than two layers (e.g., three, four, five), to enhance the mechanical properties of the prosthesis and better mimic the surrounding anatomy.

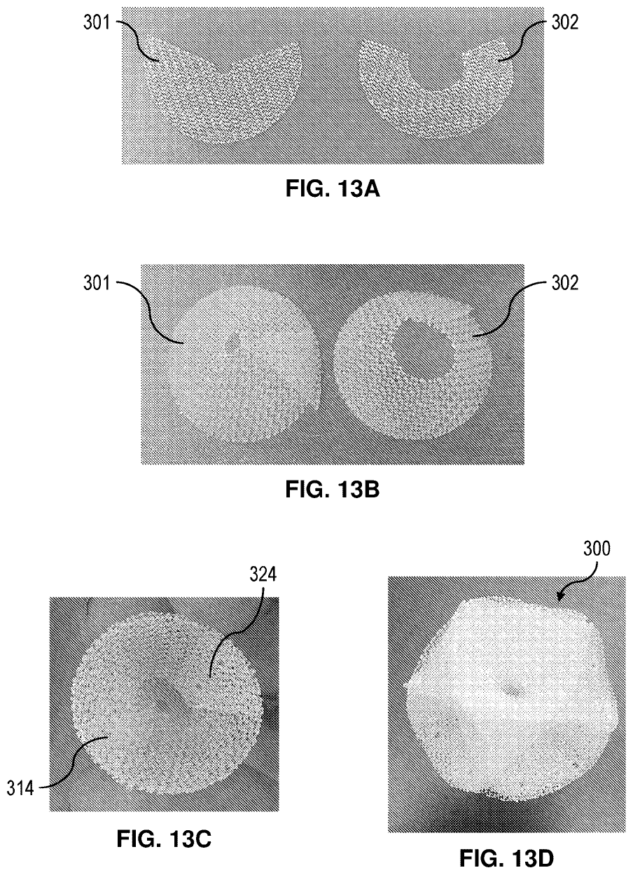

FIGS. 13A-13D show an exemplary assembly process for forming a spherical implantable prosthesis 300 formed of double layer subunits. FIG. 13A depicts two substantially two-dimensional substrates 301 and 302, which may each be manipulated to form sidewalls of a three-dimensional subunit, as shown in FIG. 13B. In some embodiments, the substrates can be formed of a porous biocompatible mesh material. As shown, the first substrate 301 may have a smaller central portion than the second substrate 302. Each substrate may have an overlapping portion, which may be formed when the substrates are manipulated into sidewalls of conical subunits. When arranged together, as shown in FIG. 13C, the overlapping portions 314 and 324 of each subunit 301 and 302, respectively, may be arranged opposite one another. Depending on the arrangements of the subunits, as well as the cutout angles, the maximum thickness of the assembled double layer subunit may be two, three, or four layers thick. Of course, embodiments in which the overlapping regions overlap or are arranged differently are also contemplated. FIG. 13D depicts an exemplary spherical prosthesis 300 formed of twelve double cone subunits. Accordingly, the prosthesis 300 includes twenty four subunits. Compared to a prosthesis formed of single cone subunits having similar geometries, material compositions, and weld point arrangements, the double cone configuration may exhibit greater compressive stiffness while still enabling tissue in-growth through its central core. As described earlier, the difference between the central portion sizes of the two cones may reduce the volume of material at the core of the prosthesis, and enhance tissue in-growth. Of course, double cone (or other subunit) arrangements formed using similar cones are also contemplated.

Table 2 below shows exemplary geometric and mechanical characterization of six spherical implantable prostheses formed with double layer subunits. Each prosthesis is designed to be generally 5 cm×5 cm×5 cm, with its double layer conical subunits including a first conical subunit having a 10% core percentage and a second conical subunit having a 22.5% core percentage.

TABLE 2
tabulated exemplary geometric and mechanical characterization of five spherical
implantable prostheses formed of double layer conical subunits.

| | Sample 1 | Sample 2 | Sample 3 | Sample 4 | Sample 5 | Sample 6 |
|---|---|---|---|---|---|---|
| Prosthesis Height (mm) | 44.68 | 46.61 | 45.75 | 46.45 | 46.52 | 45.6 |
| Prosthesis Diameter #1 (mm) | 49.85 | 50.74 | 50.13 | 49.41 | 48.88 | 52.5 |
| Prosthesis Diameter #2 (mm) | 50.29 | 50.88 | 49.35 | 50.83 | 49.6 | 52.3 |
| Force Measurement (lbf) | 3.07 | 2.66 | 3.02 | 2.84 | 3.23 | 3.21 |
| Displacement (mm) | 13.41 | 13.99 | 13.73 | 13.94 | 13.96 | 13.68 |
| Compressive Displacement | 0.3 | 0.3 | 0.3 | 0.3 | 0.3 | 0.3 |
| Cross Sectional Area (in²) | 3.05 | 3.14 | 3.01 | 3.06 | 2.95 | 3.34 |
| Normalized Force (lbf/in²) | 1.01 | 0.85 | 1 | 0.93 | 1.09 | 0.96 |
| Stiffness (psi) | 3.35 | 2.82 | 3.34 | 3.1 | 3.65 | 3.2 |

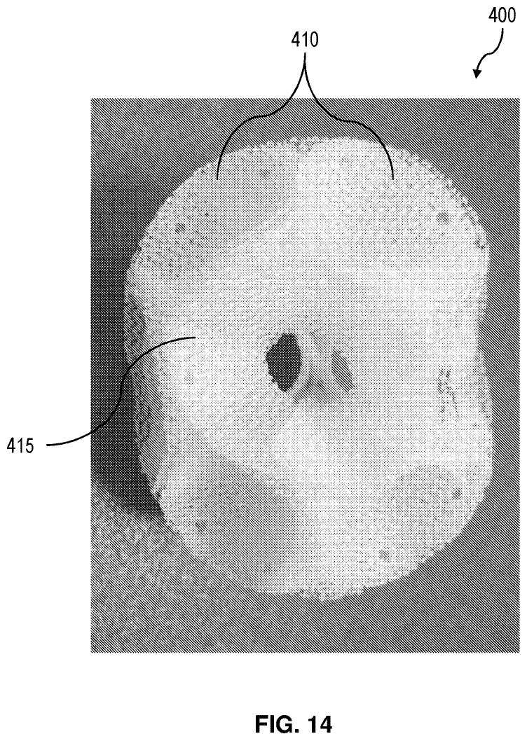

FIG. 14 depicts an exemplary ellipsoidal implantable prosthesis 400 formed of two sets of double layer subunits 410 and 415. As shown, the subunits 415 may be larger than the subunits 410, to help form the generally ellipsoidal shape. Of course, any suitable combination of subunits may be employed to form any suitable prosthesis shape, as the present disclosure is not so limited. Table 3 below shows exemplary geometric and mechanical characterization of an ellipsoidal implantable prosthesis formed with double layer subunits.

TABLE 3
tabulated exemplary geometric and mechanical characterization
of an ellipsoidal implantable prosthesis formed
of double layer conical subunits.

| | Vertical Orientation | Horizontal Orientation |
|---|---|---|
| Prosthesis Height (mm) | 52.6 | 42.3 |
| Prosthesis Width #1 (mm) | 45.8 | 52.5 |
| Prosthesis Width #2 (mm) | 42.8 | 46.2 |
| Force Measurement (lbf) | 1.17 | 1.97 |
| Displacement (mm) | 15.78 | 12.69 |
| Compressive Displacement | 0.3 | 0.3 |
| Cross Sectional Area (in²) | 2.39 | 2.95 |
| Normalized Force (lbf/in²) | 0.49 | 0.67 |
| Stiffness (psi) | 1.63 | 2.22 |

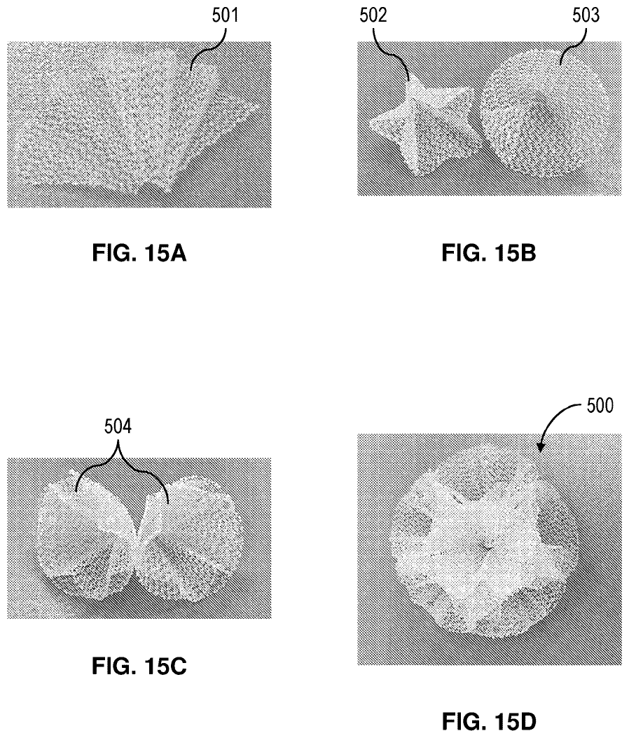

FIGS. 15A-15D depict a process of assembly for an implantable prosthesis 500 formed of a conical subunit 503 and a corrugated subunit 502. The combination of the conical shape and the corrugated shape, when overlapped (as shown in FIGS. 15C-15D) may provide larger void volumes in between the subunits, allowing for greater rates of tissue infiltration through the implantable prosthesis, while still maintain sufficient mechanical compressibility following implantation. In some embodiments, the corrugated subunit 502 may take the form of a star-shaped cone, as shown in FIG. 15B. The corrugated subunit 502 may be formed of a substrate 501 (see FIG. 15A) which may be folded or otherwise manipulated to form radial corrugations. It should be appreciated that any suitable number of radial corrugations, ranging from three (such that the subunit takes on a generally convex triangular prismatic shape) to six (such that the subunit takes on a generally hexagonal convex prismatic shape) to any other suitable number of corrugations. The substrate may then be manipulated (e.g., rolled) to form the sidewalls of a three dimensional subunit. In some embodiments, the corrugated subunit 502 may be arranged within a conical subunit 503, as shown in subunit 504 of FIG. 15C. In other embodiments, the conical subunit may be arranged within a corrugated subunit. It should be appreciated that any combination of various subunits (conical, corrugated, etc.) may be employed in any of the prostheses of the present disclosure. FIG. 15D depicts a partially assembled prosthesis 500 formed of six subunits, each of which includes at least one conical subunit and at least one corrugated subunit.

Table 4 below shows exemplary geometric and mechanical characterization of two spherical implantable prostheses having double layer subunits formed of a conical and a corrugated subunit. In the table below, sample 1 is designed to be generally 4 cm×4 cm×4 cm, and sample 2 is designed to be generally 5 cm×5 cm×5 cm.

TABLE 4
tabulate exemplary geometric and mechanical characterization of
two spherical implantable prostheses formed of double layer subunits
including a conical subunit and a corrugated subunit.

| | Sample 1 | Sample 2 |
|---|---|---|
| Prosthesis Height (mm) | 37.5 | 46.3 |
| Prosthesis Width #1 (mm) | 42.5 | 50.5 |
| Prosthesis Width #2 (mm) | 38.9 | 50.4 |
| Force Measurement (lbf) | 2.99 | 2.25 |
| Displacement (mm) | 11.25 | 13.89 |
| Compressive Displacement | 0.3 | 0.3 |
| Cross Sectional Area (in²) | 2.01 | 3.1 |
| Normalized Force (lbf/in²) | 1.49 | 0.73 |
| Stiffness (psi) | 4.95 | 2.42 |

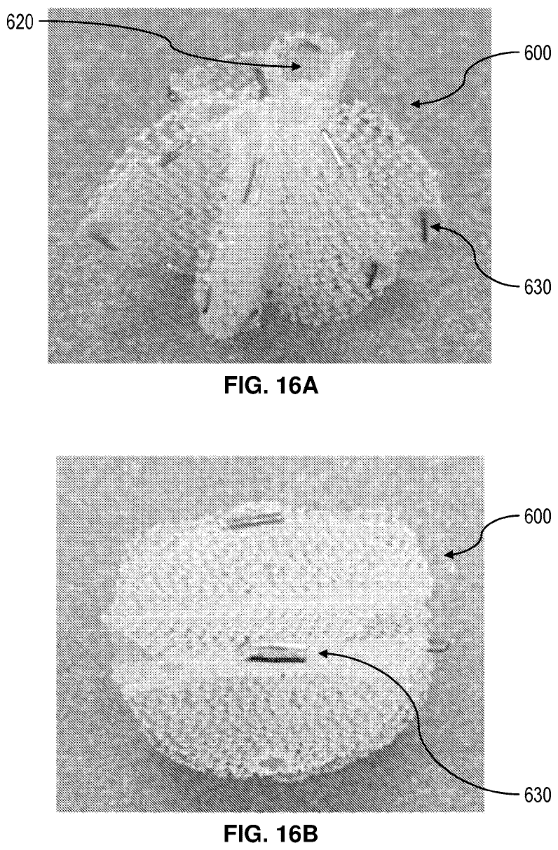

FIGS. 16A-16B depict an implantable prosthesis 600 according to some embodiments. The prosthesis 600 may be formed of two-dimensional substrates fixed to one another to form radiating fins emanating from a hollow central portion 620. The central portion 620 may serve as an empty volume for tissue infiltration. In some embodiments, the prosthesis 600 may include one or more markers 630, which may exhibit properties that may enable external detection of the prosthesis location. For example, the markers 630 may be metallic staples, which may be radiopaque and therefore distinguishable during imaging using x-ray imaging.

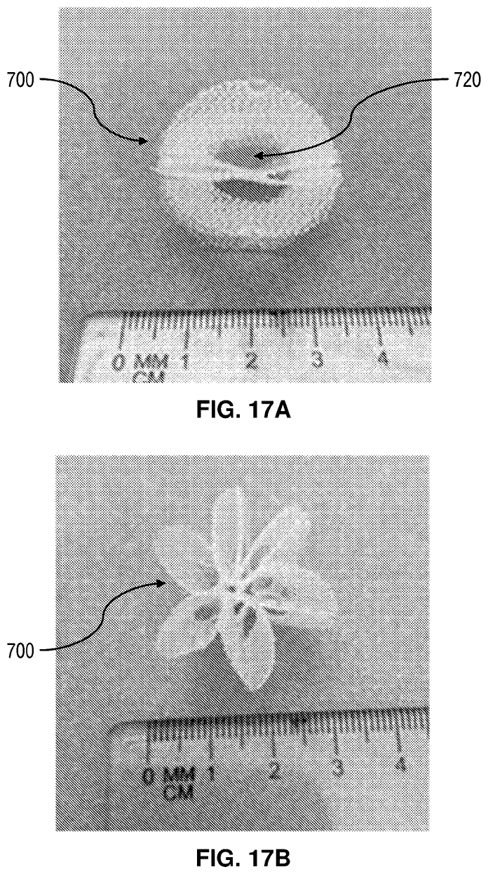

FIGS. 17A-17B depict another embodiment of an implantable prosthesis 700. The prosthesis may also include a series of radial fins formed of two-dimensional substrates. In some embodiments, the fins of prosthesis 700 may be formed of ring-shaped substrates, which may form a hollow core 720 following assembly of the prosthesis. As described relative to other embodiments, the hollow core may serve as an empty volume to enable tissue in-growth within the prosthesis following implantation.

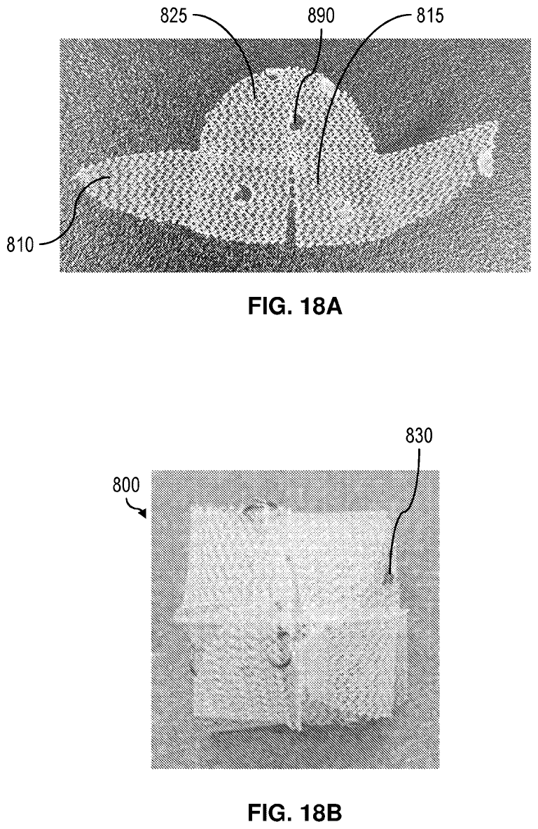

FIGS. 18A-18B depicts yet another embodiment of an implantable prosthesis 800. The prosthesis 800 may be generally cubic in shape, formed of four subunits, shown in FIG. 18A. Each subunit may be formed of various panels 810, 815, 825, which may be folded and welded together to form a quadrant of the prosthesis. Four subunits may then be welded together at weld spots 890 to form the prosthesis. The prosthesis may also include one or more markers 830, which may render the prosthesis visible within the implant site during medical imaging.

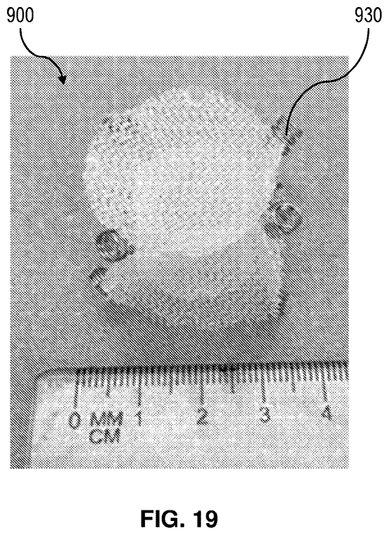

FIG. 19 depicts yet another embodiment of an implantable prosthesis 900. The prosthesis 900 may be formed of at least two generally cubic shapes, one within another. In some embodiments, the prosthesis 900 may be accompanied with markers 930 for external detection. As shown in FIG. 19, the markers may be metallic and subsequently radiopaque.

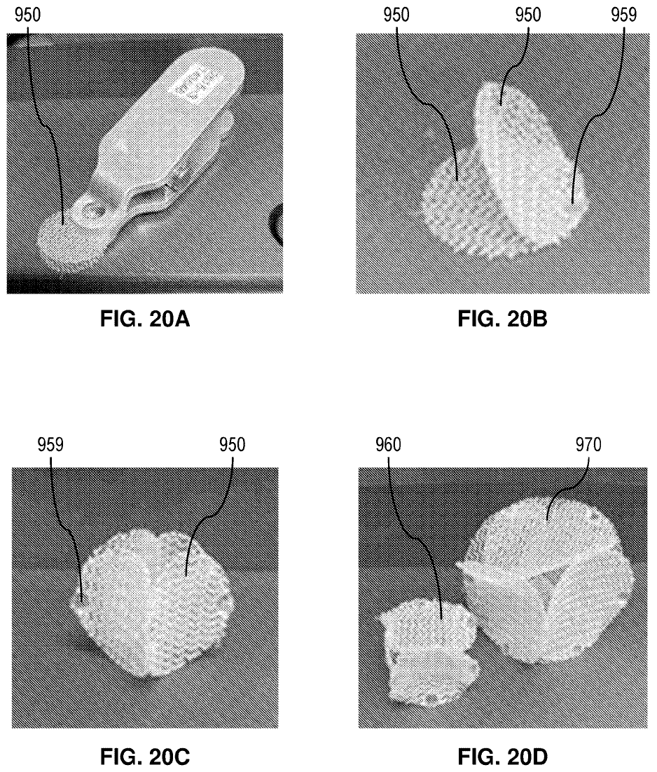

FIGS. 20A-20D depict a process of assembling the prosthesis 900 of FIG. 19. Each cubic subassembly (see cubes 960 and 970 in FIG. 20D) may be formed by fixing six two-dimensional substrates 950 together, as shown in FIGS. 20A-20B. As described earlier, the substrates 950 may be fixed to one another using welds 959, which may strengthen the prosthesis and significantly reduce the risk of deconstruction. Once both cubes are partially assembled, the smaller, inner cube 960 may be arranged within the larger, outer cube 970, as shown in FIG. 20D. The outer cube may then be closed, and one or more markers may be added to the prosthesis in preparation for implantation, as shown in FIG. 19.

It should be appreciated that any of the prostheses described herein may have any suitable shape or geometry dependent upon the application (e.g., biopsy shape and size). The prostheses may also be formed of any suitable number, size, and arrangement of subunits.

### Example 1

In some embodiments, the prostheses of the present disclosure may exhibit degradation profiles commensurate with conventional prostheses known in the art. As described previously, such degradation profiles may facilitate tissue ingrowth into the prosthesis as the prosthesis degrades, allowing the prosthesis to be ultimately replaced with natural tissue. Of course, although the prostheses described herein may exhibit conventional degradation profiles, they may exhibit improved stiffness transfer between the implant and the natural tissue to facilitate controlled tissue ingrowth.

In one example, the degradation profiles of a prosthesis is evaluated using a Swine Preclinical Lumpectomy Model over the span of twelve weeks. Table 5 below shows molecular weight retention for a prosthesis according to the present disclosure, and a prothesis known in the art. In table 5 below, sample 1 represents average data for three spherical prostheses formed of 12 conical subunits, having an overall average diameter of approximately 3 cm, and sample 2 represents average data for three conventional prostheses (e.g., PHASIX Plug and Patch).

TABLE 5
tabulated exemplary molecular weight analysis
of a spherical implantable prosthesis (sample
1) and a conventional prosthesis (sample 2).

| Time (week) | Location | Sample 1 | Sample 2 |
|---|---|---|---|
| pre-implantation | N/A | 100 | 100 |
| 4 | Center | 87 | 89 |
| | Periphery | 87 | 89 |
| 12 | Center | 66 | 65 |
| | Periphery | 67 | 64 |

As shown in table 5 above, the molecular weight analysis for samples 1 and 2 demonstrated statistically significant reduction at 4 weeks and 12 weeks, as compared to the pre-implantation state. The molecular weight analysis also demonstrated a statistically significant reduction at 12 weeks, as compared to 4 weeks post-implantation. However, the molecular weight of the center location was not statistically significantly different than that of the periphery portion for the two samples at either the 4 or 12 weeks post-implantation, which suggests a uniform molecular weight degradation through these devices.

The molecular weight analysis represented by table 5 above may suggest that the implantable prostheses described herein may exhibit the same molecular weight degradation as conventional implantable devices. However, it should be appreciated that the prostheses described herein may yield different biological responses compared to conventional prostheses, as described below relative to Example 2.

### Example 2

In some embodiments, local tissue responses following implantation of prostheses may be characterized through histological means. Specifically, local tissue responses may be characterized by the presence of neovascularization, fibrosis, collagen deposition, vascular integration, collagen morphology (via PSR staining), myofibroblasts proliferation (via SMA or smooth muscle actin staining), and neovascularization (via VWF or von Willebrand factor staining), evaluated histologically.

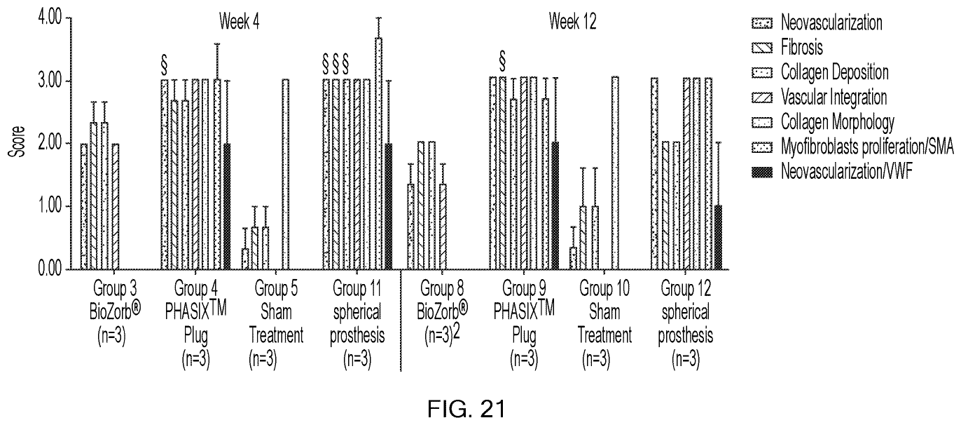

FIG. 21 shows exemplary data of the aforementioned local tissue responses for a variety of prostheses, including a commercial prosthesis BioZorb, represented by group 3 (week 4) and group 8 (week 12), a commercial prosthesis PHASIX plug, represented by group 4 (week 4) and group 9 (week 12), a control group by a sham treatment, represented by group 5 (week 4) and group 10 (week 12), and a spherical prosthesis according to the present disclosure, formed of 12 conical subunits, having an overall average diameter of approximately 3 cm, represented by group 11 (week 4) and group 12 (week 12).

The tissue response data is scored as tabulated in Table 6 below.

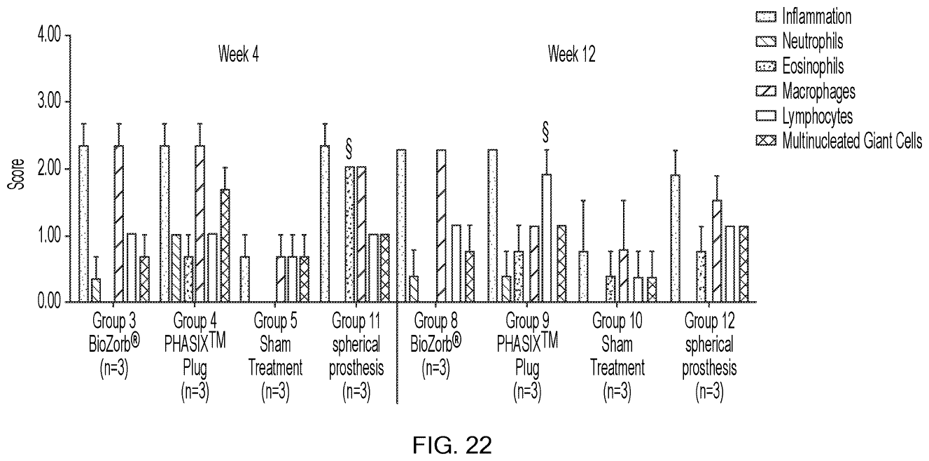

TABLE 6
observations associated with scores shown in FIGS. 21-22.

| | | Collagen Morphology | Smooth Muscle Actin (SMA) |
|---|---|---|---|
| Score | Observations Scoring Matrix | Scoring Matrix | Stain Scoring Matrix |
| 0 | No response | — | None |
| 1 | Minimal/focal/barely detectable | Green (thin) | Minimal, subtle scattered |
| | | predominant | staining |
| 2 | Mild/focal or | Mixed | Mild, notable scattered to |
| | rare multifocal/slightly detectable | | locally extensive staining |
| 3 | Moderate/multifocal | Orange-red (thick) | Moderate, multifocal to |
| | to confluent/easily detectable | predominant | regionally extensive staining |
| 4 | Marked/diffuse/overwhelming presence | — | Marked, diffuse staining. |

The exemplary data in FIG. 21 is also presented below in table 7 (week 4) and table 8 (week 12) below.

TABLE 7
observations of local tissue responses depicted in FIG. 21 at week 4.

| | Group 3 | | Group 4 | | Group 5 | | Group 11 | |
|---|---|---|---|---|---|---|---|---|
| | BioZorb ® | | PHASIX ™ Plug | | Sham Treatment | | Spherical | |
| Parameter | (n = 3) | | (n = 3) | | (n = 3) | | Prosthesis (n = 3) | |
| Neovascularization | 2.00 ± (0.00, 0.00) | 100% | 3.00 ± (0.00, 0.00) | 100% | 0.33 ± (0.58, 0.33) | 33% | 3.00 ± (0.00, 0.00) | 100% |
| | 2.00 | | | | | | | |
| Fibroplasia | 0.00 ± (0.00, 0.00) | 0% | 0.0 ± (0.00, 0.00) | 0% | 0.00 ± (0.00, 0.00) | 0% | 0.0 ± (0.00, 0.00) | 0% |
| | 0.00 | | 0.00 | | 0.00 | | 0.00 | |
| Hemorrhage | 0.00 ± (0.00, 0.00) | 0% | 0.00 ± (0.00, 0.00) | 0% | 0.00 ± (0.00, 0.00) | 0% | 0.00 ± (0.00, 0.00) | 0% |
| | 0.00 | | 0.00 | | 0.00 | | 0.00 | |
| Necrosis | 0.00 ± (0.00, 0.00) | 0% | 0.00 ± (0.00, 0.00) | 0% | 0.00 ± (0.00, 0.00) | 0% | 0.00 ± (0.00, 0.00) | 0% |
| | 0.00 | | 0.00 | | 0.00 | | 0.00 | |
| Fibrosis | 2.33 ± (0.58, 0.33) | 100 | 2.67 ± (0.58, 0.33) | 100 | 0.67 ± (0.58, 0.33) | 67% | 3.00 ± (0.00, 0.00) | 100 |
| | 2.00 | | 3.00 | | 1.00 | | 3.00 | |
| Collagen Deposition | 2.33 ± (0.58, 0.33) | 100% | 2.67 ± (0.58, 7 0.33) | 100% | 0.67 ± (0.58, 0.33) | 67% | 3.00 ± (0.00, 0.00) | 100% |
| | 2.00 | | 3.00 | | 1.00 | | 3.00 | |
| Vascular Integration | 2.00 ± (0.00, 0.00) | 100% | 3.00 ± (0.00, 0.00) | 100% | 0.00 ± (NA, NA) | 0% | 3.00 ± (0.00, 0.00) | 100% |
| | 2.00 | | 3.00 | | 0.00 | | 3.00 | |
| Collagen Morphology | NA ± (NA, NA) | NA | 3.00 ± (0.00, 0.00) | 100% | 3.00 ± (0.00, 0.00) | 100% | 3.00 ± (0.00, 0.00) | 100% |
| | NA | | 3.00 | | 3.00 | | 3.00 | |
| Myofibroblasts | NA ± (NA, NA) | NA | 3.00 ± (1.00, 0.58) | 100% | NA ± (NA, NA) | NA | 3.67 ± (0.58, 0.33) | 100% |
| proliferation/SMA | NA | | 3.00 | | NA | | 4.00 | |
| Neovascularization/ | NA ± (NA, NA) | NA | 2.00 ± (1.73, 1.00) | 67% | 0.00 ± (NA, NA) | 0% | 2.00 ± (1.73, 1.00) | 67% |
| VWF | NA | | 3.00 | | 0.00 | | 3.00 | |

Data presented as Mean ± (SD, SEM), Median, and Incidence (%).
NA = not applicable.

TABLE 8
observations of local tissue responses depicted in FIG. 21 at week 12.

| | Group 8 | | Group 9 | | Group 10 | | Group 12 | |
|---|---|---|---|---|---|---|---|---|
| | BioZorb ® | | PHASIX ™ Plug | | Sham Treatment | | Spherical Prothesis | |
| Parameter | (n = 3) | | (n = 3) | | (n = 3) | | (n = 3) | |
| Neovascularization | 1.33 ± (0.58, 0.33) | 100% | 3.00 ± (0.00, 0.00) | 100% | 0.33 ± (0.58, 0.33) | 33% | 3.00 ± (0.00, 0.00) | 100% |
| | 1.00 | | 3.00 | | 0.00 | | 3.00 | |
| Fibroplasia | 0.00 ± (0.00, 0.00) | 0% | 0.00 ± (0.00, 0.00) | 0% | 0.00 ± (0.00, 0.00) | 0% | 0.00 ± (0.00, 0.00) | 0% |
| | 0.00 | | 0.00 | | 0.00 | | 0.00 | |
| Hemorrhage | 0.00 ± (0.00, 0.00) | 0% | 0.00 ± (0.00, 0.00) | 0% | 0.00 ± (0.00, 0.00) | 0% | 0.00 ± (0.00, 0.00) | 0% |
| | 0.00 | | 0.00 | | 0.00 | | 0.00 | |
| Necrosis | 0.00 ± (0.00, 0.00) | 0% | 0.00 ± (0.00, 0.00) | 0% | 0.00 ± (0.00, 0.00) | 0% | 0.00 ± (0.00, 0.00) | 0% |
| | 0.00 | | 0.00 | | 0.00 | | 0.00 | |
| Fibrosis | 2.00 ± (0.00, 0.00) | 100% | 3.00 ± (0.00, 0.00) | 100% | 1.00 ± (1.00, 0.58) | 67% | 2.00 ± (0.00, 0.00) | 100% |
| | 2.00 | | 3.00 | | 1.00 | | 2.00 | |
| Collagen Deposition | 2.00 ± (0.00, 0.00) | 100% | 2.67 ± (0.58, 0.33) | 100% | 1.00 ± (1.00, 0.58) | 67% | 2.00 ± (0.00, 0.00) | 100% |
| | 2.00 | | 3.00 | | 1.00 | | 2.00 | |
| Vascular Integration | 1.33 ± (0.58, 0.33) | 100% | 3.00 ± (0.00, 0.00) | 100% | 0.00 ± (NA, NA) | 0% | 3.00 ± (0.00, 0.00) | 100% |
| | 1.00 | | 3.00 | | 0.00 | | 3.00 | |
| Collagen Morphology | NA ± (NA, NA) | NA | 3.00 ± (0.00, 0.00) | 100% | 3.00 ± (NA, NA) | 100% | 3.00 ± (0.00, 0.00) | 100% |
| | NA | | 3.00 | | 3.00 | | 3.00 | |
| Myofibroblasts | NA ± (NA, NA) | NA | 2.67 ± (0.58, 0.33) | 100% | NA ± (NA, NA) | NA | 3.00 ± (0.00, 0.00) | 100% |
| proliferation/SMA | NA | | 3.00 | | NA | | 3.00 | |
| Neovascularization/ | NA ± (NA, NA) | NA | 2.00 ± (1.73, 1.00) | 67% | 0.00 ± (0.00, 0.00) | 0% | 1.00 ± (1.73, 1.00) | 33% |
| VWF | NA | | 3.00 | | 0.00 | | 0.00 | |

Data presented as Mean ± (SD, SEM), Median, and Incidence (%).
NA = not applicable.

The data presented in FIG. 21 and tables 7 and 8 indicates a few statistically significant differences at 4 weeks for neovascularization (n=2), fibrosis (n=1) and collagen deposition (n=1) and one statistically significant difference for fibrosis at 12 weeks. At Week 4, mean neovascularization was statistically significantly higher (likely consequent to the scant presence of inflammation/tissue responses in sham sites as expected) in PHASIX™ Plug (group 4) when compared to Sham sites (group 5) and statistically significantly lower (likely consequent to the scant presence of inflammation/tissue responses in sham sites as expected) in Sham sites (group 5) when compared to the prosthesis of the present disclosure (group 11). At Week 4, mean fibrosis was statistically significantly lower (likely consequent to the scant presence of inflammation/tissue responses in sham sites as expected) in Sham sites (group 5) when compared to the prosthesis of the present disclosure (group 11). At Week 4, mean collagen deposition was statistically significantly lower (likely consequent to the scant presence of inflammation/tissue responses in sham sites as expected) in Sham sites (Group 5) when compared to the prosthesis of the present disclosure (group 11). Furthermore, at Week 12, mean fibrosis was statistically significantly higher (likely consequent to the scant presence of inflammation/tissue responses in sham sites as expected) in PHASIX™ Plug (Group 9) when compared to Sham sites (Group 10). These statistically significant differences may be interpreted to be biologically insignificant and likely consequent to comparison of groups with presence of devices and groups with sham sites having scant presence of inflammation/tissue responses as expected.

In some embodiments, cellular responses following implantation of prostheses may be characterized through observation of inflammation and inflammatory cell types through histology. FIG. 22 shows exemplary data of the aforementioned inflammatory responses for a variety of prostheses, including a commercial prosthesis BioZorb, represented by group 3 (week 4) and group 8 (week 12), a commercial prosthesis PHASIX plug, represented by group 4 (week 4) and group 9 (week 12), a control group by a sham treatment, represented by group 5 (week 4) and group 10 (week 12), and a spherical prosthesis according to the present disclosure, formed of 12 conical subunits, having an overall average diameter of approximately 3 cm, represented by group 11 (week 4) and group 12 (week 12). The inflammatory data is score as noted in Table 6.

The exemplary data in FIG. 22 is also presented below in table 9 (week 4) and table 10 (week 12) below.

TABLE 9
observations of inflammation depicted in FIG. 22 at week 4.

| | Group 3 | | Group 4 | | Group 5 | | Group 11 | |
|---|---|---|---|---|---|---|---|---|
| | BioZorb ® | | PHASIX ™ Plug | | Sham Treatment | | Spherical Prosthesis | |
| Parameter | (n = 3) | | (n = 3) | | (n = 3) | | (n = 3) | |
| Inflammation | 2.33 ± (0.58, 0.33) | 100% | 2.33 ± (0.58, 0.33) | 100% | 0.67 ± (0.58, 0.33) | 67% | 2.33 ± (0.58, 0.33) | 100% |
| | 2.00 | | 2.00 | | 1.00 | | 2.00 | |
| Neutrophils | 0.33 ± (0.58, 0.33) | 33 | 1.00 ± (0.00, 0.00) | 100 | 0.00 ± (0.00, 0.00) | 0% | 0.00 ± (0.00, 0.00) | 0% |
| | 0.00 | | 1.00 | | 0.00 | | 0.00 | |
| Eosinophils | 0.00 ± (0.00, 0.00) | 0% | 0.67 ± (0.58, 0.33) | 67% | 0.00 ± (0.00, 0.00) | 0% | 2.00 ± (0.00, 0.00) | 100% |
| | 0.00 | | 1.00 | | 0.00 | | 2.00 | |
| Macrophages | 2.33 ± (0.58, 0.33) | 100% | 2.33 ± (0.58, 0.33) | 100% | 0.67 ± (0.58, 0.33) | 67% | 2.00 ± (0.00, 0.00) | 100% |
| | 2.00 | | 2.00 | | 1.00 | | 2.00 | |
| Lymphocytes | 1.00 ± (0.00, 0.00) | 100% | 1.00 ± (0.00, 0.00) | 100% | 0.67 ± (0.58, 0.33) | 67% | 1.00 ± (0.00, 0.00) | 100% |
| | 1.00 | | 1.00 | | 1.00 | | 1.00 | |
| Multinucleated | 0.67 ± (0.58, 0.33) | 67% | 1.67 ± (0.58, 0.33) | 100% | 0.67 ± (0.58, 0.33) | 67% | 1.00 ± (0.00, 0.00) | 100% |
| Giant Cells | 1.00 | | 2.00 | | 1.00 | | 1.00 | |
| Plasma Cells | 0.00 ± (0.00, 0.00) | 0% | 0.00 ± (0.00, 0.00) | 0% | 0.00 ± (0.00, 0.00) | 0% | 0.00 ± (0.00, 0.00) | 0% |
| | 0.00 | | 0.00 | | 0.00 | | 0.00 | |

Data presented as Mean ± (SD, SEM), Median, and Incidence (%).

TABLE 10
observations of inflammation depicted in FIG. 22 at week 12.

| | Group 8 | | Group 9 | | Group 10 | | Group 12 | |
|---|---|---|---|---|---|---|---|---|
| | BioZorb ® | | PHASIX ™ Plug | | Sham Treatment | | Spherical Prosthesis | |
| Parameter | (n = 3) | | (n = 3) | | (n = 3) | | (n = 3) | |
| Inflammation | 2.00 ± (0.00, 0.00) | 100% | 2.00 ± (0.00, 0.00) | 100% | 0.67 ± (1.15, 0.66) | 33% | 1.67 ± (0.58, 0.33) | 100% |
| | 2.00 | | 2.00 | | 0.00 | | 2.00 | |
| Neutrophils | 0.33 ± (0.58, 0.33) | 33% | 0.33 ± (0.58, 0.33) | 33% | 0.00 ± (0.00, 0.00) | 0% | 0.00 ± (0.00, 0.00) | 0% |
| | 0.00 | | 0.00 | | 0.00 | | 0.00 | |
| Eosinophils | 0.00 ± (0.00, 0.00) | 0% | 0.67 ± (0.58, 0.33) | 67% | 0.33 ± (0.58, 0.33) | 33% | 0.67 ± (0.58, 0.33) | 67% |
| | 0.00 | | 1.00 | | 0.00 | | 1.00 | |
| Macrophages | 2.00 ± (0.00, 0.00) | 100% | 1.00 ± (0.00, 0.00) | 100% | 0.67 ± (1.15, 0.66) | 33% | 1.33 ± (0.58, 0.33) | 100% |
| | 2.00 | | 1.00 | | 0.00 | | 1.00 | |
| Lymphocytes | 1.00 ± (0.00, 0.00) | 100% | 1.67 ± (0.58, 0.33) | 100% | 0.33 ± (0.58, 0.33) | 33% | 1.00 ± (0.00, 0.00) | 100% |
| | 1.00 | | 2.00 | | 0.00 | | 1.00 | |
| Multinucleated | 0.67 ± (0.58, 0.33) | 67% | 1.00 ± (0.00, 0.00) | 100% | 0.33 ± (0.58, 0.33) | 33% | 1.00 ± (0.00, 0.00) | 100% |
| Giant Cells | 1.00 | | 1.00 | | 0.00 | | 1.00 | |
| Plasma Cells | 0.00 ± (0.00, 0.00) | 0% | 0.00 ± (0.00, 0.00) | 0% | 0.00 ± (0.00, 0.00) | 0% | 0.00 ± (0.00, 0.00) | 0% |
| | 0.00 | | 0.00 | | 0.00 | | 0.00 | |

Data presented as Mean = (SD, SEM), Median, and Incidence (%).

As shown in FIG. 22 and in tables 9 and 10, histologically, within implanted test and control sites, overall inflammation was similar between prosthesis and control implanted sites at both time points (4 weeks and 12 weeks), with overall inflammation decreasing over time in all groups except for Sham treated sites where overall inflammation was lower (as expected) than test and control treated sites at both time points. Inflammatory infiltrate was heterogeneous at both time points and at 4 weeks it was composed of neutrophils (not seen in Sham Treatment sites and in sites associated with prostheses of the present disclosure), eosinophils (not seen in Sham Treatment sites and in BioZorb® sites), macrophages, lymphocytes and multinucleated giant cells. At 12 weeks, inflammatory infiltrate was composed of neutrophils (only noted in small numbers in BioZorb® and PHASIX™ Plug sites), eosinophils (not seen in BioZorb® sites), macrophages, lymphocytes, and multinucleated giant cells. Two statistically significant differences were noted for eosinophils at 4 weeks (BioZorb® and Sham when compared to the prosthesis of the present disclosure) and one statistically significant difference was noted for lymphocytes at 12 weeks (PHASIX™ Plug when compared to Sham). All 3 statistically significant differences were interpreted to be biologically insignificant and likely consequent to comparison of groups with two different materials composing the devices and to comparison of groups with presence of devices and groups with sham sites having scant presence of inflammation/tissue responses as expected.

### Example 3

As described previously, in some embodiments, implantable prostheses may be formed of porous two-dimensional substrates, which may be cut and assembled into three-dimensional prostheses. In some embodiments, the substrates may be mesh-like sheets with porosity. For example, the substrates may be formed of PHASIX porous material, having at least two pore size distributions, a major pore size, and a minor pore size. The porosity of the prostheses may be evaluated using a statistical six-sigma system, as tabulated in Table 11 below. The table summarizes porosity data from fifteen exemplary mesh substrates used to form implantable prostheses, including number of samples N, mean pore size, standard error SE of the mean, standard deviation SD, minimum size of the distribution, first quartile Q1 porosity, median porosity, third quartile Q3 porosity, and maximum size. The porosity of the substrates tabulated in table 11 below are measured through optical characterization methods.

TABLE 11
tabulated exemplary porosity properties of mesh-like substrates used to form implantable prostheses.

| Variable | N | Mean | SE | SD | Min | Q1 | Median | Q3 | Max |
|---|---|---|---|---|---|---|---|---|---|
| Minor Pore Size (microns) | 15 | 355.51 | 5.27 | 20.41 | 326.10 | 333.30 | 354.60 | 374.90 | 384.20 |
| Major Pore Size | 15 | 777.37 | 7.50 | 29.06 | 736.80 | 754.00 | 770.90 | 810.50 | 830.70 |
| (microns) | | | | | | | | | |

In some embodiments, the implantable prostheses may be sutured to the implant site. In some embodiments, nearby conical subunits may be sutured to one another during assembly. Accordingly, the mesh-like sheets may have a suture pullout strength sufficient to withstand said forces during implantation and/or assembly. The suture pullout strength of the mesh-like substrates may be evaluated using a statistical six-sigma system, as tabulated in Table 12 below. The table summarizes suture pullout strength data from fifteen exemplary mesh substrates used to form implantable prostheses in both a machine direction (MD) and a cross direction (CD). The data in table 12 includes number of samples N, mean strength, standard error SE of the mean, standard deviation SD, minimum strength of distribution, first quartile Q1 strength, median strength, third quartile Q3 strength, and maximum strength. The strength of the substrates tabulated in table 12 below are measured by conventional tensile techniques using mechanical testing machines.

TABLE 12
tabulated exemplary suture pullout strength properties of mesh-like substrates
used to form implantable prostheses, in the machine direction (MD) and the cross direction (CD).

| Variable | N | Mean | SE | SD | Min | Q1 | Median | Q3 | Max |
|---|---|---|---|---|---|---|---|---|---|
| Suture Pullout MD (lbf) | 15 | 10.327 | 0.443 | 1.715 | 7.670 | 9.7140 | 10.210 | 11.830 | 13.530 |
| Suture Pullout CD (lbf) | 15 | 9.043 | 0.323 | 1.250 | 7.790 | 7.910 | 8.530 | 9.630 | 12.040 |

### Example 4

In some embodiments, an implantable prosthesis may be sufficiently compressible such that it may be inserted into an implant site through an incision smaller than an average size of the prosthesis. For example, a generally spherical implantable prosthesis having an average diameter of 2 cm may need to be inserted through an incision site of approximately 1.5 cm (to minimize scarring and incision formation), while retaining the majority of its size at the implant site, which may be larger than the incision site.

The size recovery of an implantable prosthesis may be evaluated along one or more directions, where the percent difference is calculated through a measurement of a dimension D prior to insertion and following insertion, as follows:

$$\% Difference = ( \frac{{D}_{{pre}_{measurement}} - {D}_{{post}_{measurement}}}{{D}_{{pre}_{measurement}}} ) * 100$$

The percent difference may be calculated for any suitable dimension, such as a height of a prosthesis, and first and second widths (see FIG. 11B). Table 13 below summarizes percent difference of heights and first and second widths for fifteen exemplary spherical implantable prostheses formed of 12 conical subunits, using a statistical six-sigma system, as described previously. The exemplary prostheses measured in table 13 below have an average diameter of 2 cm, and inserted through an incision of 1.5 cm.

TABLE 13
tabulated exemplary effect of insertion on implantable prostheses as determined by geometric changes in the prosthesis size.

| Variable | N | Mean | SE | SD | Min | Q1 | Median | Q3 | Max |
|---|---|---|---|---|---|---|---|---|---|
| Height Difference | 15 | 1.074 | 0.247 | 0.956 | −0.797 | 0.507 | 0.971 | 1.835 | 2.935 |
| Width 1 Difference | 15 | 0.685 | 0.278 | 1.077 | −1.050 | 0.112 | 0.508 | 1.247 | 3.413 |
| Width 2 Difference | 15 | 0.167 | 0.340 | 1.318 | −2.509 | −0.324 | 0.122 | 0.725 | 2.595 |

In some embodiments, the recoverability of an implantable prosthesis may be evaluated using a compression test. For example, the implantable prosthesis may be subjected to 30% compression and returned to its uncompressed state. In some embodiments, a design requirement for such a compression may be that the prosthesis return to within 10% of its original dimension following compression. Table 14 below summarizes percent difference of heights and first and second widths for fifteen exemplary spherical implantable prostheses to fulfill such requirements. The prostheses are formed of 12 conical subunits, and the tabulated values in table 14 are evaluated using a statistical six-sigma system, as described previously.

TABLE 14
tabulated exemplary recoverability after 30% compression for spherical implantable prostheses having an average
diameter of 2, 3, 4, and 5 cm, as indicated.

| Variable | N | Mean | SE | SD | Min | Q1 | Median | Q3 | Max |
|---|---|---|---|---|---|---|---|---|---|
| | Spherical prostheses with an average diameter of 2 cm | | | | | | | | |
| Height | 15 | 1.087 | 0.158 | 0.613 | 0.000 | 0.659 | 1.131 | 1.597 | 2.008 |
| Difference | | | | | | | | | |
| Width 1 | 15 | −0.061 | 0.303 | 1.174 | −1.800 | −0.758 | −0.261 | 0.184 | 3.185 |
| Difference | | | | | | | | | |
| Width 2 | 15 | 0.044 | 0.353 | 1.365 | −2.601 | −0.438 | −0.220 | 0.618 | 2.683 |
| Difference | | | | | | | | | |
| | Spherical prostheses with an average diameter of 3 cm | | | | | | | | |
| Height | 15 | 0.342 | 0.354 | 1.370 | −0.690 | −0.077 | 0.038 | 0.231 | 5.162 |
| Difference | | | | | | | | | |
| Width 1 | 15 | −0.072 | 0.176 | 0.682 | −1.301 | −0.630 | −0.006 | 0.325 | 1.377 |
| Difference | | | | | | | | | |
| Width 2 | 15 | −0.161 | 0.366 | 1.416 | −2.076 | −1.219 | −0.020 | 0.223 | 4.004 |
| Difference | | | | | | | | | |
| | Spherical prostheses with an average diameter of 4 cm | | | | | | | | |
| Height | 15 | 0.4833 | 0.0975 | 0.3777 | −0.0574 | 0.2853 | 0.4313 | 0.5387 | 1.5528 |
| Difference | | | | | | | | | |
| Width 1 | 15 | 0.376 | 0.496 | 1.919 | −1.659 | −1.133 | 0.220 | 1.417 | 5.783 |
| Difference | | | | | | | | | |
| Width 2 | 15 | −1.015 | 0.497 | 1.927 | −3.837 | −3.115 | −0.521 | 0.104 | 2.592 |
| Difference | | | | | | | | | |
| | Spherical prostheses with an average diameter of 5 cm | | | | | | | | |
| Height | 15 | 0.628 | 0.126 | 0.488 | 0.024 | 0.254 | 0.478 | 0.832 | 1.654 |
| Difference | | | | | | | | | |
| Width 1 | 15 | 0.0444 | 0.0613 | 0.2374 | −0.2177 | −0.1371 | −0.0330 | 0.1908 | 0.6045 |
| Difference | | | | | | | | | |
| Width 2 | 15 | 0.0562 | 0.0451 | 0.1746 | −0.1780 | −0.0863 | 0.0785 | 0.1777 | 0.4622 |
| Difference | | | | | | | | | |

### Example 5

In some embodiments, an implantable prosthesis may be sufficiently mechanically stiff to confer strength to the nearby tissue during implantation, but may transfer the load to the natural tissue during the re-growth process. In some embodiments, the prosthesis stiffness may be better matched with natural tissue, allowing for smoother and more controllable load transfer during the prosthesis degradation. Accordingly, in some embodiments, prostheses may be designed to have stiffness requirements, such as a supportive stiffness greater than or equal to 1.8 psi at 30% compression, and a compressive stiffness less than or equal to 6.1 psi, as a highly stiff prosthesis may invoke undesirable cellular responses. Table 15 below summarizes stiffnesses for fifteen sets of exemplary spherical implantable prostheses formed of 12 conical subunits, each having a different average diameter, as indicated. The prostheses of table 15 fulfill the aforementioned design requirements using a statistical six-sigma system, as described previously.

TABLE 15
tabulated exemplary stiffnesses of spherical implantable prostheses having different average diameters.

| Variable | N | Mean | SE | SD | Min | Q1 | Median | Q3 | Max |
|---|---|---|---|---|---|---|---|---|---|
| Stiffness (psi) | 15 | 4.083 | 0.126 | 0.488 | 3.280 | 3.640 | 4.030 | 4.520 | 4.840 |
| 2 cm diameter | | | | | | | | | |
| spherical | | | | | | | | | |
| prosthesis | | | | | | | | | |
| Stiffness (psi) | 15 | 3.2427 | 0.0562 | 0.2177 | 2.8500 | 3.0700 | 3.3200 | 3.4000 | 3.5700 |
| 3 cm diameter | | | | | | | | | |
| spherical | | | | | | | | | |
| prosthesis | | | | | | | | | |
| Stiffness (psi) | 15 | 4.0567 | 0.0643 | 0.2492 | 3.6300 | 3.8800 | 3.9700 | 4.3200 | 4.4600 |
| 4 cm diameter | | | | | | | | | |
| spherical | | | | | | | | | |
| prosthesis | | | | | | | | | |
| Stiffness (psi) | 15 | 3.0973 | 0.0605 | 0.2344 | 2.8100 | 2.9000 | 3.0600 | 3.2000 | 3.6600 |
| 5 cm diameter | | | | | | | | | |
| spherical | | | | | | | | | |
| prosthesis | | | | | | | | | |

Similar stiffness characterizations may be made for the non-spherical implantable prostheses described herein, such as ellipsoidal prostheses, as described relative to FIGS. 8A-10E. Table 16 below summarizes stiffnesses for two sets of exemplary ellipsoidal implantable prostheses formed of 14 subunits, each having a different size, as indicated. The tabulated stiffnesses have been evaluated using a statistical six-sigma system, as described previously. The specific geometries of the various ellipsoidal prostheses evaluated in table 16 are shown in table 1 above.

TABLE 16
tabulated exemplary stiffnesses of ellipsoidal implantable prostheses having different average diameters.

| Variable | N | Mean | SE | SD | Min | Q1 | Median | Q3 | Max |
|---|---|---|---|---|---|---|---|---|---|
| | Ellipsoidal prostheses with an average height of 3 cm and an average width of 2 cm | | | | | | | | |
| Horizontal | 3 | 4.003 | 0.147 | 0.254 | 3.710 | 3.710 | 4.140 | 4.160 | 4.160 |
| Stiffness (psi) | | | | | | | | | |
| Vertical Stiffness | 3 | 3.827 | 0.140 | 0.242 | 3.640 | 3.640 | 3.740 | 4.100 | 4.100 |
| (psi) | | | | | | | | | |
| | Ellipsoidal prostheses with an average height of 4 cm and an average width of 3 cm | | | | | | | | |
| Horizontal | 5 | 3.622 | 0.127 | 0.284 | 3.140 | 3.400 | 3.670 | 3.820 | 3.880 |
| Stiffness (psi) | | | | | | | | | |
| Vertical Stiffness | 5 | 3.640 | 0.106 | 0.237 | 3.280 | 3.440 | 3.660 | 3.830 | 3.930 |
| (psi) | | | | | | | | | |
| | Ellipsoidal prostheses with an average height of 5 cm and an average width of 4 cm | | | | | | | | |
| Horizontal | 3 | 3.780 | 0.189 | 0.327 | 3.410 | 3.410 | 3.900 | 4.030 | 4.030 |
| Stiffness (psi) | | | | | | | | | |
| Vertical Stiffness | 3 | 3.457 | 0.185 | 0.320 | 3.110 | 3.110 | 3.520 | 3.740 | 3.740 |
| (psi) | | | | | | | | | |

### Example 6

In some embodiments, the prostheses described herein may be tissue infiltratable, which may allow the prostheses to be fixed in place following implantation. The ingrowth of natural tissue may serve to limit the migration of the prosthesis. Prosthesis migration may be verified through tracking of a radiopaque marker associated with the prosthesis, allowing an external imaging system to view and track the prosthesis without the need for an invasive procedure. As described previously, markers may be employed to mark the specific physiological locations (e.g., tumor bed locations) within the patient for follow-up imaging to monitor recovery and potential recurrences. In some cases, it may be desirable to limit migration of the marker to within 1 cm of its original attachment site on the device following 5 months after implantation. Table 17 below summarizes migration of radiopaque markers for thirty exemplary markers between their initial implantation and five months after implantation, using a statistical six-sigma system, as described previously. The data presented in Table 17 reflects measurements at two different locations of each marker between the aforementioned time points.

TABLE 17
tabulated exemplary stiffnesses of spherical implantable prostheses.

| Variable | N | Mean | SE | SD | Min | Q1 | Median | Q3 | Max |
|---|---|---|---|---|---|---|---|---|---|
| Measurement | 30 | 0.03150 | 0.00325 | 0.01779 | 0.00580 | 0.01706 | 0.02983 | 0.04507 | 0.06591 |
| Difference- | | | | | | | | | |
| Location 1 (cm) | | | | | | | | | |
| Measurement | 30 | 0.02959 | 0.00338 | 0.01851 | 0.00081 | 0.01256 | 0.02529 | 0.04347 | 0.06674 |
| Difference- | | | | | | | | | |
| Location 2 (cm) | | | | | | | | | |

### Example 7

In some embodiments, the stiffness of an implantable prosthesis may be determined by a variety of factors, such as the hollow core percentage, the number of connections between and within each conical subunit, the distance between connections (e.g., welds), and the overall geometry of each conical subunit. In one exemplary experiment, a Pareto analysis of the variety of factors that may determine stiffness were processed to determine which elements were most influential for achieving a desired stiffness. The analysis revealed that a hollow core percentage of 10% and a degree of overlap of approximately 650 may achieve a targeted stiffness of approximately 3.75 psi for a spherical prosthesis having an average diameter of approximately 4 cm.

### Example 8

In some embodiments, the stiffness (e.g., horizontal stiffness and vertical stiffness) of an implantable ellipsoid-shaped implantable prosthesis may be determined by a variety of factors, such as hollow core percentage, the number of connections (e.g., welds) between and within each conical subunit, the distance between connections (e.g., welds) from the edge and the overall geometry of each conical submit. In one exemplary experiment, a series of different ellipsoid-shaped prosthesis (2 cm×3 cm; 3 cm×4 cm; 4 cm×5 cm) were evaluated. Each prothesis included an assembly of side, peak and middle conical subunits having different design features (e.g., degree of overlap (cutout); hollow core percentage (HC); weld number and distance from edge) as summarized in the table below.

TABLE 18
design features of ellipsoid-shaped implantable
prostheses that were evaluated.

| Size | Side | Peak | Middle |
|---|---|---|---|
| 2 × 3 | 120° cutout | 200° cutout | 125° cutout |
| | 20% HC | 25% HC | 25% HC |
| | 1 weld at 3 mm | 1 weld at 3 mm | 1 weld at 3 mm |
| 3 × 4 | 120° cutout | 218° cutout | 120° cutout |
| | 13% HC | 24.5% HC | 10% HC |
| | 2 welds at 3 & | 2 welds at 3 & | 2 welds at 3 & |
| | 7 mm | 7 mm | 7 mm |
| 4 × 5 | 55° cutout | 120° cutout | 35° cutout |
| | 10% HC | 10% HC | 10% HC |
| | 2 welds at 3 & | 2 welds at 3 & | 2 welds at 3 & |
| | 7 mm | 7 mm | 7 mm |

The evaluation showed the following: for the 2 cm×3 cm ellipsoid-shaped prosthesis a horizontal stiffness of 4.00 psi and a vertical stiffness of 3.83 psi were obtained; for the 3 cm×4 cm ellipsoid-shaped prosthesis a horizontal stiffness of 3.62 psi and a vertical stiffness of 3.64 psi were obtained; and, for the 4 cm×5 cm ellipsoid-shaped prosthesis a horizontal stiffness of 3.46 psi and a vertical stiffness of 3.78 psi were obtained.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. An implantable prosthesis comprising:
a plurality of substantially conical mesh bodies,
wherein each of the plurality of substantially conical mesh bodies is connected to at least one other of the plurality of substantially conical mesh bodies, and wherein the substantially conical bodies are arranged to form an ellipsoid.

2. A method of forming an implantable prosthesis, the method comprising:
forming a plurality of substantially conical mesh bodies; and
connecting each of the conical mesh bodies to at least one other of the other substantially conical mesh bodies to form an ellipsoid.

3. An implantable prosthesis comprising:
a plurality of substantially conical bodies,
wherein each of the plurality of substantially conical bodies is connected to at least one other of the plurality of substantially conical bodies, and
wherein the implantable prosthesis is substantially mechanically isotropic.

4. An implantable prosthesis comprising:
a plurality of substantially conical bodies, each conical body including a sidewall defining a cone shape,
wherein the sidewall of each substantially conical body is connected to the sidewall of an of at least one other adjacent substantially conical body.

5. A method of forming an implantable prosthesis, the method comprising:
forming a plurality of substantially conical bodies, each conical body including a sidewall defining a cone shape; and
connecting the sidewall of each substantially conical body to the sidewall of an of at least one other adjacent substantially conical body.

6. The implantable prosthesis or method of any of claims 1-5, wherein the implantable prosthesis comprises a hollow core.

7. The implantable prosthesis or method of any of claims 1-6, wherein the prosthesis is configured to be sized and shaped to be arranged in a lumpectomy site.

8. The implantable prosthesis or method of any of claims 1-7, wherein the implantable prosthesis is formed at least partially of a resorbable material.

9. The implantable prosthesis or method of any of claims 1-8, further comprising one or more radiopaque markers.

10. The implantable prosthesis or method of any of claims 1-9, wherein a compressive stiffness of the implantable prosthesis is between 1 psi and 10 psi, inclusive.

11. The implantable prosthesis or method of any of claims 4-5, wherein a first portion of the sidewall of each substantially conical body is connected to a second portion of the sidewall of the same substantially conical body.

12. The implantable prosthesis or method of any of claims 4-5, wherein a first portion of the sidewall of each substantially conical body is welded to a second portion of the sidewall of the same substantially conical body.

13. The implantable prosthesis or method of any of claims 1-3 and 6-10, wherein each substantially conical body comprises a sidewall, and wherein a first portion of the sidewall of each substantially conical body is welded to a second portion of the sidewall of the same substantially conical body.

14. The implantable prosthesis or method of any of claims 1-3 and 6-10, wherein each substantially conical body comprises a sidewall, and wherein the sidewall of each substantially conical body is welded to a sidewall of an of at least one other adjacent substantially conical body.

15. The implantable prosthesis or method of any of claims 1-14, wherein at least one average diameter of the implantable prosthesis is between 2 cm and 5 cm.

16. The implantable prosthesis or method of any of claims 3-5, wherein the substantially conical bodies are arranged to form an ellipsoid.

17. The implantable prosthesis or method of any of claims 1-2 and 4-5, wherein the implantable prosthesis is substantially mechanically isotropic.

18. The implantable prosthesis or method of any of claims 1-17, wherein the substantially conical bodies are arranged to form a sphere.

19. The implantable prosthesis or method of any of claims 1-18, further comprising a second plurality of substantially conical mesh bodies geometrically distinct from the first plurality of substantially conical mesh bodies.

20. The implantable prosthesis or method of claim 6, wherein the volume of the hollow core is between 10% and 35% of the total volume of the implantable prosthesis.

21. An implantable prosthesis comprising:
a plurality of mesh bodies, wherein each mesh body is connected to another mesh body;
wherein at least some of the mesh bodies include a first mesh portion connected to a second mesh portion, wherein the first portion is arranged inside a volume defined by the second mesh portion.

22. A method of forming an implantable prosthesis, the method comprising:
forming a plurality of mesh bodies;
arranging a first mesh portion of at least some of the mesh bodies inside a volume defined by a second mesh portion;
connecting the first mesh portion to the second mesh portion; and
connecting each of the mesh bodies to another mesh body.

23. The implantable prosthesis or method of any of claims 21-22, wherein the implantable prosthesis comprises a hollow core.

24. The implantable prosthesis or method of any of claims 21-23, wherein the prosthesis is configured to be sized and shaped to be arranged in a lumpectomy site.

25. The implantable prosthesis or method of any of claims 21-24, wherein the implantable prosthesis is formed at least partially of a resorbable material.

26. The implantable prosthesis or method of any of claims 21-25, further comprising one or more radiopaque markers.

27. The implantable prosthesis or method of any of claims 21-26, wherein a compressive stiffness of the implantable prosthesis is between 1 psi and 10 psi, inclusive.

28. The implantable prosthesis or method of any of claims 21-27, wherein the first portion is welded to the second portion.

29. The implantable prosthesis or method of any of claims 21-28, wherein each mesh body comprises a sidewall, and wherein a first portion of the sidewall of each mesh body is welded to a second portion of the sidewall of the same mesh body.

30. The implantable prosthesis or method of any of claims 21-29, wherein at least one average diameter of the implantable prosthesis is between 2 cm and 5 cm.

31. The implantable prosthesis or method of any of claims 21-30, wherein the mesh bodies are arranged to form an ellipsoid.

32. The implantable prosthesis or method of any of claims 21-31, wherein the implantable prosthesis is substantially mechanically isotropic.

33. The implantable prosthesis or method of any of claims 21-32, wherein the mesh bodies are arranged to form a sphere.

34. The implantable prosthesis or method of any of claims 21-33, further comprising a second plurality of mesh bodies geometrically distinct from the first plurality of mesh bodies.

35. The implantable prosthesis or method of claim 23, wherein the volume of the hollow core is between 10% and 35% of the total volume of the implantable prosthesis.

36. The implantable prosthesis or method of any of claims 21-35, wherein each of the plurality of mesh bodies are substantially conical.

37. The implantable prosthesis or method of any of claims 21-36, wherein at least some of the plurality of mesh bodies are corrugated.

38. The implantable prosthesis or method of claim 37, wherein the corrugated mesh bodies comprise at least 5 corrugations.
